(19) United States
(12) Patent Application Publication
(10) Pub. No.: US 2026/0290547 A1
(43) Pub. Date: Sep. 24, 2026

(54) SYSTEM

(71) Applicant: SoftBank Group Corp., Tokyo (JP)

(72) Inventor: Satoshi WADA, Tokyo (JP)

(73) Assignee: SoftBank Group Corp., Tokyo (JP)

(21) Appl. No.: 19/567,453

(22) Filed: Mar. 16, 2026

(30) Foreign Application Priority Data
Mar. 19, 2025 (JP) 2025-045263

(51) Int. Cl. G16H 20/60

(52) U.S. Cl. CPC G16H 20/60

(57) ABSTRACT

A system includes a processor that is configured to provide an input interface for receiving a user's budget, a number of people, and food preferences, analyze information received via the input interface and generate a prompt sentence for instructing a generative artificial intelligence model to generate a meal plan, generate, by using the generative artificial intelligence model, a one-week meal plan based on the prompt sentence, evaluate whether the generated meal plan takes nutritional balance into account, recognize an emotion of the user and adjust the meal plan based on the recognized emotion, and order food ingredients, based on the generated meal plan, from a nearest online supermarket and cause the ordered food ingredients to be delivered to a home of the user.

### CROSS-REFERENCE TO RELATED APPLICATION

This application is based on and claims priority under 35 USC 119 from Japanese Patent Application No. 2025-045263 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional meal planning and grocery ordering systems require a user to manually search for recipes, verify nutritional balance, and place separate orders with online supermarkets. Such systems typically do not leverage generative artificial intelligence models to flexibly generate meal plans tailored to individual budgets, household sizes, and food preferences. Furthermore, conventional systems do not adequately consider a user's emotional state when proposing meal plans, and therefore cannot provide psychologically adaptive or comfort-oriented meal suggestions. As a result, the user is burdened with complex decision-making regarding menu creation, nutritional considerations, and order coordination, leading to increased time consumption and reduced user satisfaction. There is a need for a system that automatically generates nutritionally balanced meal plans for a specified period based on user conditions, takes into account the user's emotional state, and seamlessly links the generated meal plans to ordering and delivery of ingredients from an appropriate online supermarket.

### SUMMARY

In order to solve the above-described problems, a system is provided comprising a processor configured to provide an input interface for receiving a user's budget, number of people, and food preferences, to analyze information received via the input interface and generate a prompt sentence for instructing a generative artificial intelligence model to generate a meal plan, and to generate, by using the generative artificial intelligence model, a one-week meal plan based on the prompt sentence. The processor is further configured to evaluate whether the generated meal plan takes nutritional balance into account, to recognize an emotion of the user and adjust the meal plan based on the recognized emotion, and to order food ingredients, based on the generated meal plan, from a nearest online supermarket and cause the ordered food ingredients to be delivered to a home of the user. The processor may be further configured to perform ordering and delivery of the food ingredients only when the generated meal plan is approved by the user, and to analyze an emotional state of the user and optimize a proposal of the meal plan based on a result of the analysis.

The term “processor” refers to a hardware or hardware-plus-software computing unit, such as a CPU, GPU, MPU, or a combination thereof, that executes instructions to implement the functions described in the claims.

The term “input interface” refers to a hardware and/or software interface, such as a graphical user interface, web form, or application screen, that enables a user to input information including budget, number of people, and food preferences.

The term “user's budget” refers to a monetary amount specified by the user that defines an allowable expenditure for purchasing food ingredients for the meal plan, for example on a per-person or per-period basis.

The term “number of people” refers to a quantity indicating how many persons will be served by the generated meal plan.

The term “food preferences” refers to user-specified conditions or constraints relating to food, including at least likes and dislikes, allergies, dietary restrictions, nutritional targets, or calorie limits.

The term “prompt sentence” refers to text or structured data generated by the processor and supplied as an input to a generative artificial intelligence model to instruct the model regarding conditions for generating a meal plan.

The term “generative artificial intelligence model” refers to a machine learning model, such as a large language model or other generative model, that generates content, including meal plans, in response to a given prompt sentence.

The term “one-week meal plan” refers to a set of meal proposals generated for multiple days corresponding to approximately seven consecutive days, including at least one meal per day. The term “nutritional balance” refers to a condition in which the generated meal plan satisfies predetermined nutritional criteria, such as appropriate distribution of macronutrients, vitamins, and minerals, and avoidance of excessive or insufficient intake according to defined standards.

The term “emotion of the user” refers to an affective state of the user, such as happiness, sadness, stress, fatigue, or similar emotional conditions, detected or inferred from user input, behavioral data, or sensor data.

The term “adjust the meal plan based on the recognized emotion” refers to modifying at least part of the generated meal plan, such as meal types, ingredients, or cooking styles, in response to the recognized emotion of the user so as to better match the user's emotional or psychological needs.

The term “nearest online supermarket” refers to an online grocery service selected based on proximity or service area to the user's home or delivery address, determined by geographic information or predefined rules.

The term “order food ingredients” refers to transmitting, from the system to the online supermarket, order information specifying types and quantities of ingredients required to realize the generated meal plan.

The term “cause the ordered food ingredients to be delivered” refers to initiating and controlling, by the processor via communication with the online supermarket or a delivery service, a process that results in physical delivery of the ordered ingredients to the user's home.

The term “approval by the user” refers to an explicit acceptance operation performed by the user, such as pressing a confirmation button or issuing a positive instruction, indicating that the user approves the generated meal plan.

The term “analyze an emotional state of the user” refers to processing data related to the user, such as textual input, interaction history, voice, facial expressions, or physiological signals, to determine or classify the user's current emotion or mood.

The term “optimize a proposal of the meal plan” refers to selecting, adjusting, or re-generating one or more elements of the meal plan so as to better satisfy one or more criteria, including at least the user's emotional state, food preferences, nutritional balance, and budget.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

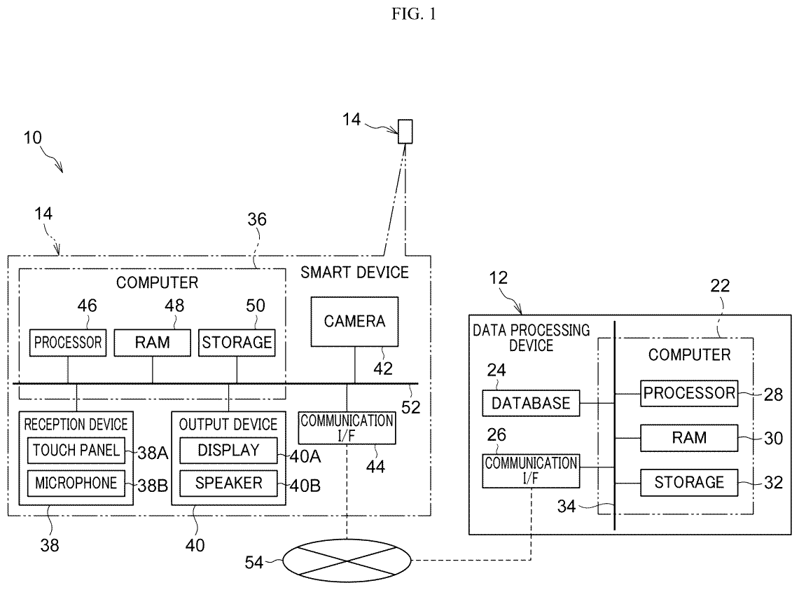

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

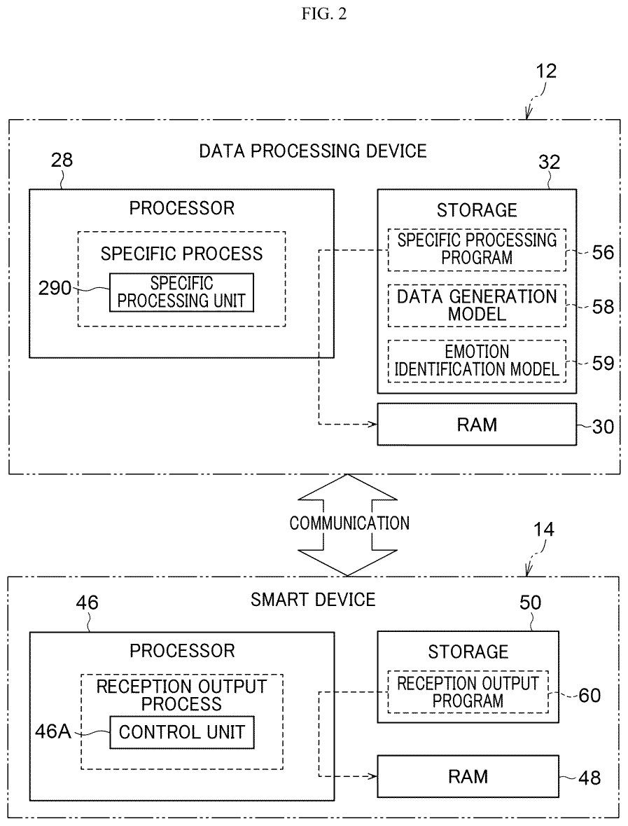

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples.

Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional meal-planning and ingredient-ordering systems mainly focus on presenting static recipe lists or preconfigured menus and rely heavily on manual user operations. In many implementations, a user is required to separately input dietary conditions, manually search candidate menus, independently evaluate nutritional balance, and then manually prepare a shopping list and place an order with an electronic commerce service. Such processing flows impose several technical problems in terms of computer technology. First, existing systems generally treat the user's condition information, free-form menu text, and ingredient data as loosely related, unstructured data. As a result, the systems cannot reliably and automatically transform a user's high-level conditions into a consistent sequence of machine-processable data objects, from user input, to a prompt for a generative AI model, to a structured ingredient list mapped to order formats for external commerce systems. This causes frequent manual intervention, error-prone data transfers, and redundant client-server communications, and leads to inefficient utilization of computing and communication resources.

Second, many systems do not provide a unified mechanism for automatically generating a prompt sentence for a generative AI model based on structured user condition information, and do not integrate the generative AI output with downstream data-processing pipelines. In particular, known systems typically do not parse the generated meal-plan text to extract and normalize ingredient information and aggregate quantities across multiple days at the server side. Consequently, a server cannot automatically synthesize a machine-readable ingredient list suitable for conversion into product-identification information and quantity information required by heterogeneous commerce back-end systems, thereby limiting automation and scalability.

Third, conventional architectures often process user approval and order generation on separate logical paths, without tightly coupling approval state with the automatic ordering pipeline. This may result in unnecessary order-preparation computations on the server or inadvertent transmission of order information prior to explicit user approval. Such architectures can degrade system reliability and waste computational resources and network bandwidth.

Fourth, typical systems do not adapt meal-plan generation to a user's emotional state in a technically integrated manner. Emotional information, if considered at all, is often handled outside the core data-processing pipeline and is not encoded in the prompt sentence provided to the generative AI model. As a result, the system cannot systematically optimize meal-plan proposals based on emotional conditions, and the server is unable to automatically influence the generative model's output in a structured, reproducible way. This leads to suboptimal personalization and inefficient use of generative AI capabilities.

Accordingly, there is a need for an improved computer-implemented system that, by means of a processor, storage device, communication interface, and user interface device, (i) acquires and stores user condition information as structured data, (ii) automatically generates a natural-language prompt sentence for a generative AI model based on that structured data, (iii) receives and analyzes generated meal-plan information to extract and aggregate ingredient information, (iv) automatically generates and transmits order information in a format required by external electronic commerce systems only upon user approval, and (v) optionally incorporates user emotional state information into the prompt sentence to optimize meal-plan proposals. Such a system should improve the technical efficiency, reliability, and integration of the entire pipeline from user input to order placement, and reduce the computational and operational overhead associated with manual or fragmented processing in conventional systems.

The specific processing by the specific processing unit 290 of the data processing device 12 in Example 1 is realized by the following means.

The present invention provides a server comprising a processor, a storage device, a communication interface, and a user interface device, the processor being configured to acquire user condition information including budget information, number-of-persons information, and dietary-preference information via the user interface device; store, in the storage device, the acquired user condition information as structured information; generate, based on the structured information, a natural-language prompt sentence for a generative information processing model; transmit, via the communication interface, the prompt sentence to the generative information processing model and receive, from the generative information processing model, meal-plan information for a plurality of days generated based on the prompt sentence; analyze the received meal-plan information to extract food-item information and quantity information and aggregate the quantity information for each food item across the plurality of meals to generate ingredient-list information; generate, based on the ingredient-list information, order information including product-identification information and quantity information in a format required by an electronic commerce service information processing apparatus; receive, via the user interface device, an approval input from a user regarding the meal-plan information; and, in response to the approval input, transmit the order information to the electronic commerce service information processing apparatus via the communication interface and store, in the storage device, order-acceptance information and delivery-schedule information received from the electronic commerce service information processing apparatus, while inhibiting generation and transmission of the order information when the approval input is not received; and optionally include, in the prompt sentence, condition information relating to an emotional state of the user so as to cause the generative information processing model to generate meal-plan information adjusted according to the emotional state of the user. This enables a computer-centered improvement in which the server automatically orchestrates a continuous, machine-processable pipeline from structured user input through prompt-sentence generation, generative AI meal-plan generation, ingredient extraction and aggregation, and conditional order generation and transmission, thereby reducing manual data handling, improving reliability and consistency of data transformations, optimizing use of network and processing resources, and enhancing personalization of meal-plan proposals based on user emotional state.

The term “user condition information” refers to information indicating one or more requirements, constraints, or preferences of a user regarding meal planning, including at least budget information, number-of-persons information, and dietary-preference information, and optionally further including temporal conditions, nutritional constraints, or allergy conditions. The term “budget information” refers to numerical or categorical information indicating an upper limit or range of expenditure permitted by a user for one or more meal plans or for associated ingredient purchases.

The term “number-of-persons information” refers to numerical information indicating a count of individuals for whom the meal plan is to be prepared.

The term “dietary-preference information” refers to information indicating likes, dislikes, restrictions, or requirements relating to food or ingredients, such as avoidance of certain food categories, allergy information, nutritional constraints, or dietary styles.

The term “user interface device” refers to one or more hardware and software components configured to present information to a user and to receive input from the user, including, for example, a display, input peripherals, and application software executing on a client device.

The term “storage device” refers to a hardware component or subsystem capable of storing data in a non-transitory manner, such as a memory device, magnetic storage, solid-state storage, or a database system.

The term “communication interface” refers to a hardware and software interface configured to send and receive data via a communication network, such as a network interface controller, modem, or wireless communication module, together with related communication protocols.

The term “structured information” refers to data that is organized according to a predefined format or schema, such as records, fields, and data types, enabling systematic processing, querying, and transformation by an information processing apparatus.

The term “prompt sentence” refers to a natural-language or semi-structured text expression generated by the server for input to a generative information processing model, the text expression encoding user condition information and, optionally, additional constraints or instructions.

The term “generative information processing model” refers to an information processing model, typically implemented as a machine learning model such as a neural network, that generates output data, including text describing meal plans, in response to input data such as a prompt sentence.

The term “meal-plan information” refers to information describing one or more meals over a period of time, including, for example, dish names, meal times, associated ingredients, and optionally nutritional characteristics.

The term “food-item information” refers to information identifying individual food items or ingredients included in one or more meals of the meal-plan information, such as names, categories, or standardized item identifiers.

The term “quantity information” refers to information indicating an amount, volume, weight, or count associated with a food item or ingredient, typically expressed in units such as grams, liters, or pieces.

The term “ingredient-list information” refers to structured information representing a list of food items and corresponding quantity information aggregated across one or more meals or days of the meal plan.

The term “order information” refers to structured information generated for transmission to an electronic commerce service information processing apparatus, the information including at least product-identification information and quantity information, and optionally including user-identification information, delivery address information, and delivery time information.

The term “product-identification information” refers to information that uniquely or distinctively identifies a product in an electronic commerce system, such as a product code, stock-keeping unit, or catalog identifier.

The term “electronic commerce service information processing apparatus” refers to an information processing system operated by or on behalf of an electronic commerce service provider and configured to receive order information, manage product data, process orders, and generate order-acceptance and delivery-schedule information.

The term “order-acceptance information” refers to information indicating that an order transmitted to an electronic commerce service information processing apparatus has been received and accepted, and may include an order identifier, confirmation status, and related metadata.

The term “delivery-schedule information” refers to information indicating an expected or scheduled time or time window for delivery of items associated with an order, and may include date, time, or time-range data.

The term “approval input” refers to user input indicating explicit acceptance or confirmation of meal-plan information, received via the user interface device and used as a condition for initiating generation and transmission of order information.

The term “emotional state of the user” refers to information representing a psychological or affective state of a user, such as stress, happiness, fatigue, or other mood-related conditions, which can be encoded as condition information for influencing meal-plan generation.

In the following embodiments, a data processing apparatus is referred to as a server, and a client device such as a smartphone, tablet, or personal computer is referred to as a terminal. A user operates the terminal to interact with the server.

The server executes application software on a hardware platform that may include a central processing unit (CPU), a graphics processing unit (GPU), a main memory, a non-volatile storage device, and a network interface. For example, the server runs on a general-purpose computing platform such as an x86_64 or ARM-based server, using an operating system such as a general-purpose server operating system and a web application framework such as a server-side framework (for example, a scripting-language framework or an object-oriented framework). The server further accesses a relational database system such as a general relational database (e.g., MySQL-type or PostgreSQL-type database) and communicates over a network such as the Internet via HTTPS.

The terminal executes a web browser or a native mobile application. The terminal includes a display, an input device such as a touch panel, and a communication module. The terminal presents graphical user interface components, such as text fields, drop-down lists, check boxes, and buttons, generated by client-side software (for example, a browser script or a native UI toolkit). The user uses the terminal to input user condition information including budget information, number-of-persons information, and dietary-preference information, and to approve or decline a generated meal plan.

The server acquires the user condition information from the terminal via the network interface. The server receives the information in a structured format (for example, a JSON-based or other structured payload) and stores the information into a storage device managed by the relational database system. The server normalizes the data into a schema that may include a user table, a request table, a meal plan table, and an order table. The server assigns primary keys and foreign keys to relate records, and stores fields such as budget, number_of_people, dietary_constraints, emotional_state, and timestamps in a normalized form. By storing user condition information as structured information rather than free text, the server enables deterministic generation of prompt sentences and reduces parsing overhead in later stages, improving data management and query efficiency.

The server generates a prompt sentence for a generative AI model based on the structured user condition information. The server uses a template engine or formatting module that constructs a natural-language string from the stored fields. For example, if the user condition information indicates a budget of 5000 units, four persons, an avoidance of meat, and an allergy to nuts, the server generates a prompt sentence such as:
“Please propose a meal plan for 4 people within a budget of 5000 yen that avoids meat and does not include any nuts.”

In another embodiment, the server includes nutritional and temporal constraints, and generates a prompt sentence such as:
“Please propose a nutritionally balanced 2-day meal plan for 3 people within a total budget of 6000 yen, avoiding seafood and dairy products, and keeping each person under 1800 kcal per day.”

In a further embodiment, the server additionally encodes an emotional state of the user into the prompt sentence. For example, when the user condition information includes an emotional state such as “stressed” or “fatigued,” the server generates a prompt sentence such as:
“Please propose a 3-day dinner meal plan for 2 people within a budget of 7000 yen that avoids spicy food and shellfish, keeps each meal under 700 kcal, and is suitable for a user who is feeling stressed and needs easy-to-digest meals.”

By using deterministic templates and rule-based mapping from structured fields to natural-language segments, the server produces prompt sentences that are consistent and machine-verifiable. This reduces ambiguity compared with free-form user prompts and improves reproducibility of generative AI outputs, which is a technical improvement in how the generative AI model is integrated into the system.

The server interfaces with a generative AI model that is implemented, for example, as a transformer-based neural network. The generative AI model is trained on large-scale text data and is configured to accept an input sequence of tokens representing the prompt sentence and to output a sequence of tokens representing generated meal-plan information. The generative AI model uses an encoder-decoder or decoder-only architecture with multiple self-attention layers, feed-forward layers, and layer normalization. During training, the generative AI model minimizes a loss function such as cross-entropy between predicted tokens and ground-truth tokens, and updates internal weight parameters using gradient descent or a variant thereof. The generative AI model may be trained with data augmentation techniques such as random masking, synonym replacement, or paraphrasing of meal descriptions and dietary constraints.

The server transmits the prompt sentence as an input sequence to the generative AI model via an application programming interface. The server specifies model parameters such as temperature, top-k sampling, or maximum output length to control diversity and length of generated text. The generative AI model generates meal-plan information, which typically includes day-level headers, meal types (breakfast, lunch, dinner), dish names, and ingredient lists with approximate quantities. For example, the generative AI model may return text such as:

### “Day 1:

Breakfast: Oatmeal with banana and honey (Ingredients: rolled oats 50 g, banana 1 piece, honey 10 g, low-fat milk 150 ml)
Lunch: Vegetable pasta with tomato sauce (Ingredients: pasta 80 g, tomato 100 g, onion 30 g, olive oil 5 g, garlic 5 g)
Dinner: Grilled tofu salad with mixed vegetables (Ingredients: tofu 100 g, lettuce 50 g, cucumber 30 g, carrot 20 g, olive oil 5 g).”

The server receives the generated meal-plan information and performs a series of data-processing operations to convert the unstructured text into structured ingredient-list information. The server uses parsing algorithms that leverage both rule-based patterns and statistical cues. For instance, the server may define regular expressions for ingredient lines, identify patterns such as “(Ingredients: . . . )”, and split the content into item-quantity pairs. The server may also maintain a dictionary or ontology of ingredient names and unit types to normalize different textual representations (e.g., “tomatoes,” “tomato,” “fresh tomato”) into a canonical food-item identifier.

The server aggregates quantity information across multiple days and meals. Internally, the server constructs a map or associative array keyed by ingredient identifiers, where each value contains a cumulative quantity in a standard unit. When the server encounters a new ingredient line, it converts the free-text quantity (“1 piece,” “100 g,” “0.5 cup”) into a normalized unit using conversion rules and adds the value to the cumulative quantity. This algorithm reduces redundant computations and avoids inconsistent manual aggregation. Because the server performs normalization and aggregation in a structured manner, the server can generate an ingredient list that is directly usable for order generation, thereby improving computational efficiency and reducing human error.

The server converts ingredient-list information into order information for an electronic commerce service. The server maps normalized ingredient identifiers to product-identification information used by the commerce platform, using a mapping table stored in the database. The mapping may include product codes or stock-keeping units, preferred brands, and package sizes. The server calculates required package counts by dividing aggregated quantities by package sizes and rounding according to predefined rules. This computation is performed algorithmically using numeric operations on the server, and it avoids ad hoc, manual estimation by the user.

The server then generates order information, including product-identification information, quantity information, user identity, delivery address, and desired delivery time. The server formats this information according to the schema required by the commerce system and transmits it via the communication interface. The commerce system responds with order-acceptance information and delivery-schedule information, which the server stores in the database and presents to the user through the terminal.

The terminal presents the generated meal-plan information and ingredient-list information to the user, together with controls that allow the user to approve or decline the proposed plan. The user may also modify some conditions and request regeneration. The server enforces a rule that order information is generated and transmitted only when an explicit approval input is received from the terminal. This coupling of approval state with the internal pipeline is implemented as a state machine in the server, where transitions to an “order-ready” state are conditioned on an approval flag stored in the database. This design reduces the risk of inadvertent ordering and eliminates unnecessary processing of order generation in the absence of user approval, which reduces computational and communication load.

In some embodiments, the server incorporates emotional-state information of the user into the generative AI process. The server may receive emotional-state information from the terminal via explicit selection (e.g., the user selects “tired,” “stressed,” or “energetic”) or via an external sensing module. The server encodes the emotional state as a categorical or vector feature and embeds it in the prompt sentence as natural-language text, as described above. Alternatively or additionally, the server may generate a structured representation of emotional state and pass it as contextual metadata to the generative AI model, influencing attention weights or biasing output probabilities through conditioning. Because the emotional state is explicitly encoded and fed into the generative model by the server, the system can reproduce and adjust its behavior across sessions, leading to more consistent and technically controlled personalization than manual human judgment.

The server improves computer technology in several ways. First, by converting user condition information into structured data and then into a systematically generated prompt sentence, the server reduces variance and ambiguity at the interface between human input and the generative AI model. This leads to more stable model outputs and reduces the need for repeated calls to the generative AI model, thereby saving computation and network bandwidth. Second, by implementing a parsing and aggregation pipeline that transforms free-text meal-plan information into normalized ingredient-list information, the server enhances data management and enables automatic conversion to commerce-ready order specifications. This replaces ad hoc manual interpretation with a deterministic, machine-checkable process, reducing error rates and improving throughput.

Third, the server uses an explicit mapping between normalized ingredient identifiers and product-identification information of a commerce platform. This mapping allows the server to perform direct numeric computations on quantities and package sizes, which is more efficient than human manual lookup. The server's data structures (e.g., relational tables and in-memory maps) and numeric algorithms enable scalable operation across many users and orders, improving performance compared with conventional systems in which each user manually constructs shopping lists.

Fourth, by coupling the user approval input to the internal state machine for order generation, the server avoids speculative or redundant computations of order information. The server only performs product mapping and quantity calculations after an approval event has been recorded, which reduces unnecessary CPU load and database writes, and thereby improves resource utilization and reduces latency.

Fifth, by integrating emotional-state information as a technical feature into the generation of prompt sentences and the generative AI conditioning, the server provides a computational mechanism to systematically change model behavior. This is different from merely letting a human subjectively alter menus. The server encodes emotional states as part of the input space of the generative model, allowing the model to adjust content generation according to a defined rule rather than ad hoc human judgment. This approach improves the predictability and controllability of generative AI behavior, which is a concrete technical improvement. In alternative embodiments, the server may use different database technologies, such as a document-oriented database or a key-value store, to persist user condition information, meal-plan information, and order information. The server may implement the parsing and normalization pipeline using a combination of deterministic rules and a secondary lightweight classifier trained to identify ingredient segments within generated text. The generative AI model may be deployed locally on the same physical server or accessed as a remote service, and may differ in size, architecture depth, or training regimen, as long as the model accepts a prompt sentence and generates meal-plan information that can be parsed by the server.

In still other embodiments, the terminal may be a dedicated appliance in a household environment, such as a smart kitchen device with an integrated display and voice interface. In such a case, the terminal captures voice input from the user, converts the voice input into text using a speech-recognition module, and transmits the resulting textual user condition information to the server. The server then performs the same structured-data conversion, prompt generation, generative AI interaction, parsing, aggregation, and order generation as described above. The system thereby adapts to different hardware configurations without changing the essential data-processing pipeline.

Because the server automatically orchestrates a complete data flow—from structured user input, to generation of a controlled prompt sentence, to generative AI model execution, to structured ingredient aggregation and order translation—the system improves the technical functioning of the computer network as a whole. Network traffic is reduced by minimizing redundant requests; storage operations are made more efficient by using normalized schemas; and processing time is reduced by deferring expensive computations until approval is obtained. The system thus goes beyond mere automation of human mental processes and constitutes an improvement in computer-based data processing and resource management.

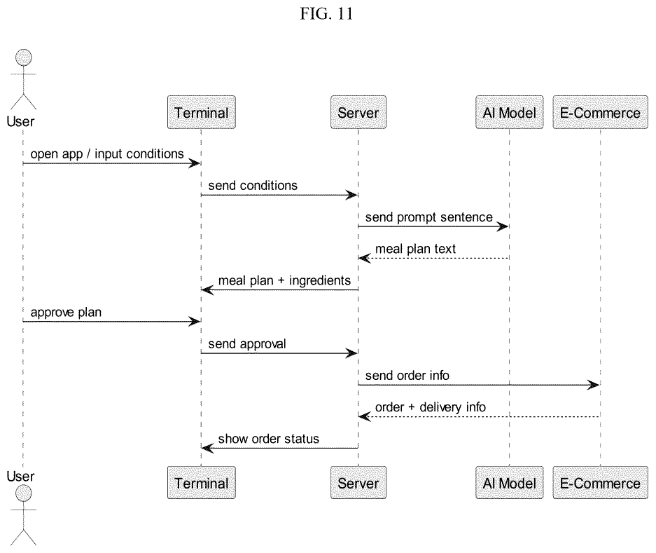

The following describes the processing flow using FIG. 11.

### Step 1:

The user operates the terminal to open an application screen for meal planning.

The terminal displays input fields for budget, number of persons, dietary preferences, and optionally emotional state, using a graphical user interface.

Input: The user enters textual and numerical values (e.g., budget=“5000”, number of persons =“4”, dietary preferences=“avoid meat, allergy: nuts”, emotional state=“stressed”).

The terminal validates the format of the input (for example, the terminal checks that budget and number of persons are numeric and that text fields do not exceed a predetermined length).

Output: The terminal generates a structured data object representing user condition information, including budget information, number-of-persons information, dietary-preference information, and optionally emotional-state information.

### Step 2:

The terminal transmits the user condition information to the server via a secure communication protocol such as HTTPS.

Input: The terminal uses the structured data object created in Step 1 as the request payload.

The terminal serializes the data (for example, in JSON format) and sends it to a predetermined server endpoint.

Output: The server receives a network request containing serialized user condition information.

### Step 3:

The server receives the network request and parses the serialized user condition information. Input: The server obtains from the communication interface a serialized payload that includes budget, number of persons, dietary preferences, and optional emotional state.

The server deserializes the payload into internal data structures (for example, fields in an object or record) and verifies that mandatory fields are present and valid.

The server writes the parsed values into a storage device by executing database operations to insert or update corresponding records in a normalized schema (for example, a request record linked to a user record).

Output: The server produces a stored record of user condition information in the database and an in-memory representation ready for further processing.

### Step 4:

The server generates a prompt sentence for the generative AI model based on the structured user condition information.

Input: The server reads from its in-memory representation or from the database the budget information, number-of-persons information, dietary-preference information, and optional emotional-state information.

The server applies a template-based generation algorithm, concatenating natural-language fragments corresponding to each field. For example, the server converts budget=“5000”, persons=“4”, preferences=“avoid meat, allergy: nuts”, emotional state=“stressed” into the prompt sentence:
“Please propose a 3-day dinner meal plan for 4 people within a total budget of 5000 yen that avoids meat and does not include any nuts, and that is suitable for a user who is feeling stressed and needs easy-to-digest meals.”

The server may apply rule-based logic to add phrases for calorie limits or nutritional balance when such constraints are present in the structured data.

Output: The server produces a natural-language prompt sentence string that encodes the user condition information in a form suitable for input to the generative AI model.

### Step 5:

The server sends the prompt sentence to the generative AI model and requests generation of meal-plan information.

Input: The server uses the prompt sentence from Step 4 and model-control parameters (such as maximum output length, temperature, or other decoding settings).

The server formats a model-request object containing the prompt sentence and parameters, and transmits this object to the generative AI model via an application programming interface.

The generative AI model, implemented as a trained transformer-based neural network, processes the prompt sentence, converts its tokens into internal vector representations, performs multiple layers of self-attention and feed-forward computation, and produces a sequence of output tokens that form meal-plan text.

Output: The server receives from the generative AI model a text sequence representing meal-plan information for multiple days and multiple meals.

### Step 6:

The server parses the received meal-plan information and converts it into structured meal data.

Input: The server obtains raw text from the generative AI model, for example, text including day labels, meal names, and ingredient lists.

The server applies parsing rules that detect line breaks, numbered lists, and keyword patterns such as “Day”, “Breakfast”, “Lunch”, “Dinner”, and “Ingredients:”.

The server splits the text into individual day segments and, within each day, splits the text into meal segments, extracting dish names and associated ingredient lines.

The server writes the structured representation of days, meals, dishes, and ingredients into internal data structures and may store this representation into a meal plan table in the database.

Output: The server produces structured meal-plan data, including day-level and meal-level records with associated ingredient lines, suitable for further computation.

### Step 7:

The server extracts ingredient items and quantities from the structured meal data and aggregates them into an ingredient list.

Input: The server uses the structured meal-plan data from Step 6, including ingredient lines such as “rolled oats 50 g” or “banana 1 piece”.

The server applies text parsing algorithms and pattern matching to separate ingredient names from quantity values and units.

The server normalizes ingredient names using an internal dictionary or mapping table (for example, mapping “tomatoes” and “tomato” to a single canonical ingredient identifier) and converts different units into a standard unit when possible (for example, converting “0.5 cup” to an equivalent number of grams using stored conversion factors).

The server accumulates quantities in an in-memory data structure keyed by canonical ingredient identifier, summing the numeric quantity values across all days and meals.

Output: The server produces ingredient-list information that consists of canonical ingredient identifiers and aggregated quantity information for each ingredient.

### Step 8:

The server generates order information for an electronic commerce service based on the aggregated ingredient list.

Input: The server reads the ingredient-list information produced in Step 7, including canonical ingredient identifiers and total required quantities.

The server consults a product-mapping table stored in the database to associate each ingredient identifier with one or more product-identification codes used by the electronic commerce platform and with package size information.

The server computes the number of packages required for each product by dividing the aggregated quantity by the package size and applying rounding rules (for example, rounding up to ensure sufficiency of ingredients).

The server then constructs order information that includes, for each product, product-identification information and quantity information (in units of packages), and also includes user-related data such as delivery address and preferred delivery time.

Output: The server produces a complete order-information data structure ready to be transmitted to the electronic commerce service information processing apparatus.

### Step 9:

The server transmits the meal-plan information and aggregated ingredient list to the terminal for user review and approval.

Input: The server uses the structured meal-plan data from Step 6 and the ingredient-list information from Step 7.

The server formats this information as a response message and sends it to the terminal through the communication interface.

The terminal receives the response, parses the structured data, and renders it on a display, showing per-day menus, meal descriptions, and a summarized list of required ingredients.

Output: The user sees, on the terminal, the generated meal-plan information and ingredient summary, along with user interface controls to approve or modify the plan.

### Step 10:

The user reviews the displayed meal plan and provides an approval input via the terminal.

Input: The user inspects the menus and ingredients on the display and interacts with the controls presented by the terminal (for example, tapping an “Approve plan” button).

The terminal captures the approval action as an event and constructs an approval message referencing the identifier of the associated meal plan or request.

The terminal sends the approval message to the server via the network interface using a predefined application protocol.

Output: The server receives a message that explicitly indicates approval status for a specific meal-plan instance.

### Step 11:

The server controls order generation based on the received approval input.

Input: The server reads the approval message from the communication interface and identifies the relevant meal-plan record and associated ingredient list.

The server updates an approval flag in the database to indicate that the user has approved the meal plan.

If the approval flag indicates approval, the server proceeds to use the order information generated in Step 8; if the approval flag does not indicate approval, the server refrains from sending any order information and may delete or suspend temporary order-related data.

Output: The server either authorizes the order-information transmission path (for an approved plan) or blocks it (for a non-approved plan), thereby determining whether an actual order will be placed.

### Step 12:

The server transmits the order information to the electronic commerce service and processes the response.

Input: The server uses the order-information data structure from Step 8, conditioned on the approval decision from Step 11.

The server formats the order information according to the external commerce API specification and sends it through the communication interface to the electronic commerce service information processing apparatus.

The electronic commerce service returns order-acceptance information and delivery-schedule information. The server receives this response and parses fields such as order identifier, acceptance status, and expected delivery date and time.

The server writes the order-acceptance and delivery-schedule information into the database and associates it with the corresponding user and meal-plan records.

Output: The server produces stored order-status records and delivery information that can be retrieved and displayed to the user on the terminal.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented meal planning systems typically rely on static rule sets or simple recommendation engines that match user profiles to predefined menus. Such systems are limited in their ability to flexibly accommodate complex, multi-dimensional constraints, such as budget, number of persons, detailed dietary preferences, nutritional constraints, and dynamic user emotional states, while also automating ordering and delivery processes from multiple vendors. As a result, conventional systems often generate meal proposals that fail to satisfy user constraints in a consistent manner, require extensive manual adjustment by the user, and do not scale well when many users or heterogeneous vendors are involved.

Moreover, existing systems that employ machine learning for content generation frequently treat the generative model as a black box that outputs unstructured text. These systems do not effectively transform the unstructured output into structured data that can be programmatically verified, optimized, and bound to downstream operations such as order generation, vendor selection, and logistics control. Consequently, such systems suffer from low reliability, difficulties in enforcing budget and nutritional constraints, and an inability to automatically reconcile generated content with the capabilities and inventory of external sales entities.

Additionally, known platforms do not adequately integrate user interaction history, selection history, and evaluation information into the generation pipeline of a generative AI model at the prompt-construction level. Instead, user preference learning and content generation are often siloed processes. This separation leads to inefficient use of computational resources, repetitive or irrelevant recommendations, and degraded user experience, because the generative model is not systematically guided by a user-specific preference pattern encoded into the prompt sentence itself.

Furthermore, conventional architectures for automated meal planning and ordering typically lack a robust feedback loop between a terminal interface and a server-side orchestration engine that coordinates prompt generation, model inference, structured data extraction, cost calculation, vendor selection, and delivery status tracking. The absence of such a loop results in fragmented workflows that require multiple manual interventions, introduce latency and errors in order processing, and fail to provide real-time transparency of order and delivery status to the user.

Therefore, there is a need for a computer-implemented system that improves the way a generative AI model is integrated into a meal planning and ordering workflow, by (i) systematically generating and regenerating prompt sentences based on validated structured input data, nutritional evaluations, cost calculations, and learned user preferences, (ii) transforming model outputs into structured data suitable for automated downstream processing, and (iii) tightly coupling the model-driven planning with vendor selection and logistics control. Such improvements constitute an enhancement of the functioning of the computer system itself, enabling more efficient, accurate, and adaptive processing of complex constraints in a networked meal planning and ordering environment.

The specific processing by the specific processing unit 290 of the data processing device 12 in Application Example 1 is realized by the following means.

The present invention provides a server comprising a processor configured to provide, to a terminal, an input interface for receiving structured data including a user budget, a number of persons, dietary preferences, and nutritional constraints, to acquire the structured data from the terminal, to analyze the structured data and generate a prompt sentence including conditions corresponding to the user budget, the number of persons, the dietary preferences, and the nutritional constraints, to input the prompt sentence to a generative AI model and cause the generative AI model to perform numerical computation to generate a meal plan for a predetermined period, to extract structured information from the generated meal plan, to calculate indices related to nutritional balance, energy intake, ingredient composition, and cost, to evaluate whether the generated meal plan satisfies predetermined nutritional and budget conditions, to regenerate or update the prompt sentence based on the evaluation so as to enforce the conditions, to incorporate user emotional state information and learned user preference patterns into the regenerated prompt sentence, to re-input the regenerated prompt sentence to the generative AI model so as to adapt meal plan generation to each user, to configure an ingredient list corresponding to an approved meal plan, to select a sales entity based on user location information and delivery conditions, to transmit order data of ingredients to an external information processing apparatus of the selected sales entity, to receive order status and delivery status from the external information processing apparatus, and to notify the terminal of the statuses only when an approval input of the meal plan is received from the terminal. This enables improved computer functionality for meal planning and ordering by transforming unstructured generative outputs into verifiable structured data, by iteratively controlling the generative AI model through dynamically constructed prompt sentences that encode multi-dimensional constraints and user-specific preferences, and by seamlessly integrating the model-driven planning with automated vendor selection and logistics control in a unified server-side workflow.

The term “terminal” refers to an information processing device, such as a smartphone, tablet, personal computer, or similar user-operated device, that provides a user interface for data input, display of generated meal plans, and interaction with a server over a communication network.

The term “server” refers to an information processing apparatus, typically including one or more processors and memory, that executes software programs to receive data from terminals, perform analysis and computation, interact with external systems, and return responses to the terminals.

The term “processor” refers to one or more hardware processing units, such as a central processing unit or a graphics processing unit, and associated control circuitry, capable of executing instructions to perform the functions described in the claims, including data analysis, prompt generation, model inference control, and communication management. The term “structured data” refers to data organized according to a predefined schema or format, such as a key-value structure or tabular representation, which is suitable for programmatic parsing, validation, and processing by the server.

The term “user budget” refers to information indicating a monetary range or limit specified by a user for costs associated with a meal plan and corresponding ingredients.

The term “number of persons” refers to information indicating how many individuals are intended to be covered by a generated meal plan.

The term “dietary preferences” refers to user-specified conditions or choices regarding types of food or dietary patterns, including but not limited to vegetarian, vegan, low-sodium, or allergy-related restrictions.

The term “nutritional constraints” refers to limitations or targets relating to nutritional properties of meals, including, for example, maximum or minimum values of calories, macronutrients, or other nutritional indicators.

The term “prompt sentence” refers to a machine-readable textual instruction or query generated by the server, which includes one or more conditions or requirements and is provided as input to a generative AI model to control the content and structure of the model's output.

The term “generative AI model” refers to a machine learning model, such as a neural network-based generative model, configured to produce text or structured data outputs in response to an input prompt sentence, by performing numerical computations on internal parameters and input representations.

The term “meal plan” refers to a set of proposed meals for a predetermined period, such as a plurality of days, including information concerning dishes, meal categories, and optionally ingredients and nutritional values.

The term “predetermined period” refers to a fixed time span for which a meal plan is generated, such as a day, multiple days, a week, or another defined interval.

The term “nutritional balance” refers to a state in which the composition of a meal plan satisfies predetermined criteria related to distribution and quantity of nutrients, such as energy, protein, fat, and carbohydrates, according to one or more nutritional guidelines.

The term “energy intake” refers to the total or per-meal caloric value associated with food items in a meal plan.

The term “ingredient composition” refers to the set and proportion of individual food ingredients included in a meal plan or in a particular meal.

The term “indices related to nutritional balance, energy intake, and ingredient composition” refers to numerical or categorical indicators calculated from a meal plan that quantify aspects of nutrition, caloric content, and ingredient usage for evaluation and comparison.

The term “user emotional state” refers to an estimated or provided indication of a user's affective condition, such as stress level, mood, or preference for comfort food, which may influence adjustment of a meal plan.

The term “user preference pattern” refers to a representation of user-specific tendencies or likes, derived from historical user behavior and feedback, and used to guide or personalize the generation of meal plans.

The term “input history information” refers to records of user-provided input parameters, such as past budgets, dietary preferences, and constraints, accumulated over time.

The term “selection history information” refers to records of which meal plans, dishes, or options a user has selected, approved, or rejected in past interactions.

The term “evaluation information” refers to user feedback data, including ratings, comments, or explicit evaluations regarding generated meal plans or individual dishes.

The term “sales entity” refers to a commercial organization, such as a store, vendor, or distribution center, that supplies ingredients and is capable of receiving electronic orders and arranging delivery.

The term “distribution base” refers to a physical or logical facility associated with a sales entity from which ordered ingredients are prepared and dispatched for delivery to users.

The term “external information processing apparatus” refers to a computer system operated by a sales entity or logistics provider, which communicates with the server to receive order data, report processing status, and provide delivery information.

The term “order data” refers to structured information specifying what ingredients are to be supplied, in which quantities, and under what delivery conditions, including user address and desired delivery time.

The term “ingredient list” refers to a set of ingredient entries derived from a generated meal plan, each entry specifying a type of ingredient and a quantity required to prepare the proposed meals.

The term “order status” refers to information indicating a current processing stage of an order, including states such as accepted, preparing, shipped, or completed.

The term “delivery status” refers to information indicating a current stage of physical transportation of ingredients, including states such as in transit, out for delivery, or delivered.

The term “approval input” refers to input data provided by the user via the terminal indicating acceptance of a proposed meal plan, permitting the system to proceed with ordering and delivery operations.

The term “modification input” refers to input data provided by the user via the terminal indicating changes or adjustments to a proposed meal plan, such as replacing dishes, altering quantities, or modifying constraints.

The term “text analysis processing” refers to one or more computational techniques applied to text, including parsing, pattern matching, and extraction of entities or attributes, to transform unstructured model outputs into structured data.

The term “data structure” refers to an organized arrangement of data elements, such as lists, arrays, records, or objects, that allows efficient access, modification, and computation in a program.

The term “budget range” refers to an interval defined by a lower and/or upper monetary bound within which the total cost of ingredients associated with a meal plan is intended to remain.

The term “numerical computation” refers to operations such as vector and matrix calculations, nonlinear transformations, and probabilistic estimations performed by a machine learning model to generate output from input data.

The term “communication network” refers to a wired or wireless infrastructure, such as the Internet or a mobile data network, that enables data transmission between the terminal, the server, and external information processing apparatuses.

In one embodiment, the server includes at least one processor, a memory, and a communication interface connected via an internal bus. The server executes a computer program stored in the memory. The computer program is implemented, for example, using an interpreted or compiled language such as Python and is executed in an application framework such as a web application framework. The server communicates with at least one terminal over a communication network such as the Internet using standard protocols such as HTTPS over TCP/IP.

The terminal includes a processor, a memory, a display unit, and an input unit such as a touch panel or keyboard. The terminal executes an application, which may be a native application or a web browser application, that presents a graphical user interface to the user. The terminal transmits structured data to the server and receives responses from the server through a communication module.

The server stores, in the memory, a plurality of software modules including at least: a request reception module, a validation module, a prompt generation module, a generative AI model interface module, a post-processing module, a preference learning module, a vendor selection module, and a status management module. The server additionally stores model parameters and auxiliary data structures used by a generative AI model, which is implemented using a numerical computation library such as a tensor computation framework.

The generative AI model is implemented as a neural network having a sequence processing architecture, for example a transformer architecture including an input embedding layer, a plurality of self-attention layers, feed-forward layers, normalization layers, and an output projection layer. The model processes tokenized text sequences corresponding to prompt sentences and generates text sequences representing meal plans. The model parameters include weight matrices and bias vectors that are updated during a training phase and fixed during an inference phase in the deployed system.

The server uses a tokenizer module to convert prompt sentences into sequences of token identifiers. The tokenizer module applies a subword segmentation algorithm such as byte-pair encoding or a similar method to map characters and words into a fixed vocabulary. The server represents the token sequences as multidimensional arrays (tensors) stored in the memory. The generative AI model interface module passes these tensors to the neural network for inference. The neural network performs numerical computation, including matrix multiplications, non-linear activation functions, and attention score calculations, to compute probability distributions over possible next tokens. The server applies a decoding algorithm such as beam search or top-k sampling to select output token sequences from the probability distributions.

The server uses the request reception module to acquire, from the terminal, structured data including a user budget, a number of persons, dietary preferences, and nutritional constraints. The terminal presents input fields and selection controls to the user and encodes the user's input into a structured format such as a key-value mapping. The terminal transmits the structured data to the server via the communication interface. The server stores the received data in a transient data structure in the memory and performs validation using the validation module. The validation module checks ranges, data types, and required fields and normalizes values where appropriate.

The server uses the prompt generation module to generate a prompt sentence based on the validated structured data. The prompt generation module reads the budget, number of persons, dietary preferences, and nutritional constraints from memory and inserts these values into a text template. The prompt generation module may, for example, construct a prompt sentence such as:
“Please propose a vegetarian menu for two people for one week with a budget of 3,000 yen and no more than 500 kcal per meal, and for each day, please provide the name of the dish for breakfast, lunch, and dinner, a brief description, and the necessary ingredients and approximate portions in an easy-to-understand text format, not JSON format.”

In another example, the server constructs a prompt sentence in English such as: “The user's budget is 3000 yen for 2 persons. Please generate a 7-day vegetarian meal plan where each meal is 500 kcal or less. For each day, list breakfast, lunch, and dinner with dish names, brief descriptions, required ingredients, and approximate quantities in plain text format.”

The server optionally adds meta-instructions to the prompt sentence to specify output structure, such as explicit line breaks or delimiters to facilitate later parsing by the post-processing module. By embedding detailed structural constraints directly into the prompt sentence, the server reduces the complexity of subsequent parsing and increases the reliability of converting unstructured output into structured data. This contributes to improved computational efficiency and reduced error rate in the end-to-end meal planning workflow. The generative AI model interface module supplies the generated prompt sentence to the neural network and controls inference parameters such as maximum output length, temperature, and sampling strategy. The server adjusts these parameters based on system load or required precision. For example, the server may reduce beam width in low-latency scenarios to decrease inference time or increase beam width in high-precision scenarios to improve the quality of the meal plan generation. By dynamically controlling decoding parameters, the server optimizes trade-offs between processing speed and output diversity, representing an improvement in the functioning of the computer system rather than a mere automation of human decision-making.

The server receives the text output from the generative AI model and stores it in the memory. The post-processing module applies text analysis processing to the output. The post-processing module uses pattern matching, delimiters, and context-based parsing to extract entities such as dish names, meal categories (e.g., breakfast, lunch, dinner), ingredient names, quantities, and nutritional information when present. The post-processing module converts the extracted data into explicit data structures, such as lists of records, where each record includes fields for day, meal category, dish name, ingredients, quantity, and nutritional indicators. The server uses this explicit data representation to compute indices related to nutritional balance, energy intake, ingredient composition, and total cost.

The server uses a nutritional evaluation submodule to compare computed nutritional indicators with predefined nutritional constraints. The nutritional evaluation submodule reads reference values and threshold values from a nutritional guideline database stored in the memory. The submodule calculates, for each day and for the overall meal plan, metrics such as total daily calories, distribution of macronutrients, and presence of restricted ingredients. The server uses these metrics to evaluate whether the generated meal plan complies with the nutritional constraints specified by the user and the predefined guidelines.

The server uses a cost calculation submodule to estimate the cost of the ingredients required for the meal plan. The cost calculation submodule accesses price data stored in a database or received from sales entities. The server associates each ingredient with a unit price and multiplies the unit price by the required quantity to compute a total cost for each ingredient and for the entire meal plan. The server compares the total cost with the user budget. When the total cost exceeds the budget or the nutritional constraints are not satisfied, the server regenerates or updates the prompt sentence.

The prompt generation module, in cooperation with the nutritional evaluation and cost calculation submodules, constructs a revised prompt sentence that, for example, states: “The total cost of the ingredients in the menu just suggested exceeds your budget. Please reconfigure your weekly vegetarian menu to stay within your budget of ¥3,000, prioritizing lower priced ingredients while maintaining less than 500 kcal per meal.” By including explicit feedback about cost and nutritional violations in the revised prompt sentence, the server guides the generative AI model to search a different region of its learned parameter space that is more likely to satisfy the constraints. This iterative prompt regeneration and model inference form a control loop that enforces multi-dimensional constraints in a way that would be computationally burdensome for manual rule-based systems. The control loop enhances computational efficiency by converging more quickly toward feasible meal plans, reduces the number of failed plans, and improves resource utilization on the server.

The server further uses a preference learning module to learn user preference patterns. The preference learning module stores input history information, selection history information, and evaluation information in a user profile database. The server computes statistical features for each user, such as frequency of acceptance of particular dish types, avoidance of certain ingredients, or typical calorie range. The server may also employ an auxiliary machine learning model, such as a smaller neural network or a matrix factorization model, to map these features into a vector representation of the user's preferences.

The server incorporates the learned preference vector into the prompt sentence by adding descriptive text reflecting the user's historical choices. For example, the prompt generation module may extend the prompt sentence with text such as:
“Users have in the past preferred Japanese flavors and vegetable-based menu choices, and tend to avoid spicy dishes. Please take this preference into account when proposing a menu.” By translating numeric preference vectors into prompt text, the server uses the generative AI model in a non-conventional way, where the prompt sentence encodes an implicit user-specific distribution over outputs. This mechanism improves the accuracy of personalization and reduces the need for explicit rule tuning, thereby enhancing the performance of the computer system in generating relevant meal plans at scale.

The server also processes user emotional state information. The terminal may provide an interface where the user selects a current mood or stress level, or the server may infer an emotional state from usage patterns. The server encodes the emotional state into textual instructions in the prompt sentence, for example:
“Since users are currently under high stress, preference should be given to menus that are easy on digestion and require fewer cooking steps.”

This information modifies the constraints and ranking criteria applied by the generative AI model through the prompt. As a result, the model's internal attention and probability distributions shift toward recipes that match the emotional context. The integration of emotional state and learned preference patterns into the prompt sentence forms a novel technique for conditioning the generative AI model that is different from conventional static prompts or simple parameter tuning, thereby improving output relevance and reducing computational waste on unsuitable candidate plans.

The vendor selection module in the server uses the structured ingredient list and the user's location information to select a suitable sales entity. The server stores information about available sales entities, including geographic coverage, inventory availability, delivery options, and pricing. The vendor selection module applies an algorithm that minimizes a cost function combining distance, expected delivery time, ingredient availability, and price. The server may implement this as a multi-criteria optimization process and uses index structures in the database to efficiently query candidate vendors. By converting the generative model's textual output into structured ingredient lists, the server is able to programmatically match required ingredients with vendor inventories. This direct coupling between AI-generated content and structured vendor data yields improved data management and reduces communication overhead by sending only relevant order data to the selected external information processing apparatus.

The terminal displays the generated meal plan and the ingredient list to the user. The terminal renders a user interface containing textual descriptions of dishes, ingredient lists, and nutritional information. The user reviews the proposed meal plan and provides an approval input or a modification input. The terminal sends the user's response to the server. The server executes the order data transmission and the delivery instruction only after receiving the approval input. This gating mechanism prevents unnecessary orders and reduces network traffic and computational load on external vendor systems.

The server transmits order data to the selected sales entity's external information processing apparatus using an application programming interface. The order data includes the ingredient list, quantities, delivery address, and delivery time preferences. The sales entity's system processes the order and returns status updates. The server stores order status and delivery status in the memory and updates them as new information is received. The status management module in the server converts internal status codes into user-readable messages and sends these messages to the terminal. The terminal displays the status to the user, enabling real-time tracking of the meal-related logistics.

From a technical perspective, the described configuration improves computer technology in several ways. The integration of iterative prompt generation, constraint evaluation, and generative AI model control improves processing accuracy and reduces the number of iterations needed to obtain a valid meal plan. The explicit transformation of unstructured generative outputs into structured data allows the server to perform deterministic computations for nutritional evaluation, cost calculation, vendor matching, and logistics control, which enhances the reliability and predictability of the system. The use of user preference patterns and emotional state information encoded at the prompt level optimizes the generative AI model's search process in its parameter space, thereby reducing computational redundancy and shortening response time.

The server's modular architecture, with distinct but interacting modules for validation, prompt generation, model inference, post-processing, preference learning, and vendor selection, provides a clear data flow and allows for parallelization and caching strategies. For example, the server can cache frequently used ingredient price data and nutritional reference data to reduce external queries, and can reuse partial preference computations across multiple sessions for the same user. These techniques reduce communication load, improve throughput, and lower latency.

Various modifications and alternative embodiments are possible. In one variation, the generative AI model may use a recurrent neural network or a hybrid architecture instead of a transformer. In another variation, the nutritional evaluation submodule may incorporate additional health metrics such as sodium or fiber targets. In another variation, the vendor selection module may consider dynamic traffic information or real-time inventory levels provided by the sales entities. In yet another variation, the preference learning module may incorporate collaborative filtering based on anonymized data from multiple users to refine individual preference vectors.

In all of these embodiments, the server, the terminal, and the generative AI model cooperate through explicit data structures, prompt sentences, and numerical computation processes to implement a technically grounded, computer-implemented meal planning and ordering system that goes beyond a mere automation of human tasks and achieves improved computational efficiency, accuracy, and data management in a networked environment.

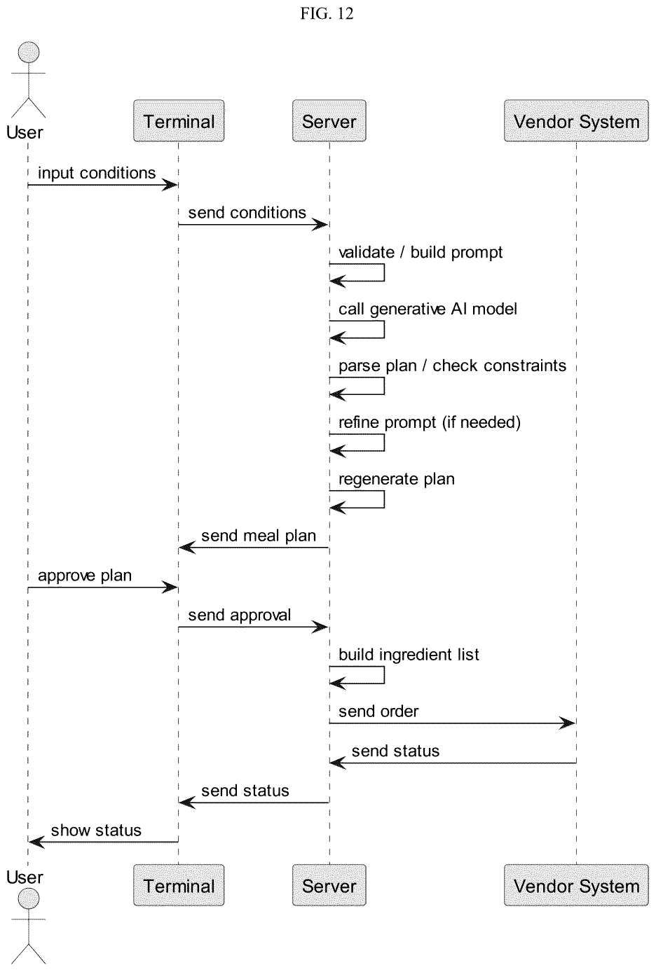

The following describes the processing flow using FIG. 12.

### Step 1:

The terminal presents an input interface to the user and initializes input data.

The terminal displays input fields for a user budget, a number of persons, dietary preferences, and nutritional constraints using UI components such as text boxes, selection lists, and sliders.

The user operates the terminal to enter a numeric value for the budget, select the number of persons, choose dietary preferences (for example, vegetarian or low-carb), and specify nutritional constraints (for example, calories per meal or per day).

The terminal takes these user inputs as raw values (input) and performs local validation, such as checking that the budget is non-negative and that the number of persons is at least one.

The terminal converts the validated values into structured data, such as key-value pairs stored in an internal data object (output), and prepares the data for transmission to the server.

### Step 2:

The terminal transmits structured user data to the server.

The terminal takes the structured data object created in Step 1 as input and serializes it into a format suitable for network communication, such as a text-based representation.

The terminal performs data processing by encoding characters into a network-safe representation and attaching metadata such as a destination endpoint and authentication information.

The terminal outputs a network request containing the serialized data and sends the request over a communication network to the server, using a secure transport protocol.

### Step 3:

The server receives and validates the structured user data.

The server takes the network request from the terminal as input and decodes the serialized data to reconstruct the structured data object in server memory.

The server processes the data by performing validation checks on each field, including type checking, range checking, and completeness verification.

The server may normalize units (for example, converting calories to a standard unit) or map textual dietary preferences to internal category codes.

The server outputs a validated and normalized structured data object that is stored in working memory for further processing.

### Step 4:

The server generates a primary prompt sentence for the generative AI model based on the structured data.

The server takes the validated structured data object as input and retrieves the user budget, number of persons, dietary preferences, and nutritional constraints from the object.

The server performs string processing by inserting these values into a text template, applying concatenation operations and conditional branch logic to include or omit specific clauses depending on which constraints are present.

The server outputs a prompt sentence in natural language, such as:
“The user's budget is 3000 yen for 2 persons. Please generate a 7-day vegetarian meal plan where each meal is 500 kcal or less. For each day, list breakfast, lunch, and dinner with dish names, brief descriptions, required ingredients, and approximate quantities in plain text format.”

### Step 5:

The server tokenizes the prompt sentence and prepares input tensors for the generative AI model.

The server takes the prompt sentence from Step 4 as input and applies a tokenizer that splits the sentence into tokens based on subword units.

The server converts each token into a token identifier by looking up the token in a vocabulary table stored in memory.

The server organizes the sequence of token identifiers into a numerical array and pads or truncates the sequence to a fixed length, thereby creating an input tensor representation.

The server outputs an input tensor and associated attention masks that are passed to the generative AI model as numerical input.

### Step 6:

The server executes inference on the generative AI model to generate a raw meal plan.

The server takes the input tensor and attention masks from Step 5 as input and feeds them into a neural network implementing the generative AI model.

The server performs numerical computation, including matrix multiplications, non-linear activation functions, and attention weight calculations, layer by layer through the model.

The server produces, at each decoding step, a probability distribution over possible next tokens and applies a decoding algorithm such as beam search or top-k sampling to select the most suitable next token.

The server repeats this computation for multiple steps, using previously generated tokens as additional input, until an end-of-sequence condition is met.

The server outputs a sequence of generated token identifiers representing a raw text description of a meal plan.

### Step 7:

The server decodes the generated tokens into a textual meal plan.

The server takes the sequence of generated token identifiers from Step 6 as input and maps each identifier back to a corresponding subword or word using the vocabulary table.

The server concatenates the decoded tokens into a continuous text string, inserting appropriate spaces and line breaks.

The server outputs a textual meal plan, which may include day labels, meal categories, dish names, ingredient descriptions, and cooking hints, as a human-readable text block in memory.

### Step 8:

The server transforms the textual meal plan into structured meal plan data.

The server takes the textual meal plan from Step 7 as input and applies text analysis processing to identify structural elements.

The server performs pattern matching and segmentation based on keywords such as “Day 1,” “Breakfast,” and “Ingredients,” and extracts dish names, ingredients, quantities, and, if present, nutritional values.

The server constructs a structured data representation, such as a list of records where each record includes fields for day, meal category, dish name, ingredient list, and nutritional indicators.

The server outputs a structured meal plan object that is suitable for deterministic computation and evaluation.

### Step 9:

The server computes nutritional and cost indices for the structured meal plan.

The server takes the structured meal plan object from Step 8 as input and iterates through each record to aggregate nutritional values and ingredient quantities.

The server accesses a nutritional database and a price database to map ingredients to nutritional values and unit prices.

The server performs arithmetic operations, such as summing calories per meal and per day, calculating nutrient ratios, and summing costs per ingredient and for the total plan.

The server outputs numerical indices including total daily calories, macronutrient distributions, and total cost, along with flags indicating whether the plan meets the nutritional constraints and budget.

### Step 10:

The server evaluates constraint satisfaction and decides whether to regenerate a prompt sentence.

The server takes the numerical indices and constraint flags from Step 9 as input and compares them with the user's budget and nutritional constraints.

The server executes conditional logic to determine whether any constraint is violated, such as a total cost exceeding the budget or daily calories exceeding a specified limit.

The server outputs a decision value, for example a Boolean flag or status code, indicating either acceptance or the need for adjustment.

### Step 11:

The server generates an adjusted prompt sentence when constraints are not satisfied.

The server takes the decision value from Step 10 and the constraint violation details as input and constructs an updated prompt sentence that explicitly describes the violations and requests modifications.

The server performs string processing to embed instructions such as “replace expensive ingredients with cheaper alternatives” or “reduce calorie content while preserving dietary preferences” into the new prompt sentence.

The server outputs an adjusted prompt sentence, such as:
“The previously generated meal plan exceeds the user's budget of 3000 yen. Please regenerate a 7-day vegetarian meal plan that remains within the 3000 yen budget while keeping each meal under 500 kcal and prioritizing lower-cost ingredients.”

### Step 12:

The server integrates user emotional state and preference patterns into the prompt sentence.

The server takes the user's emotional state information and learned preference vector from a preference database as input.

The server translates the numeric preference vector and emotional state into descriptive text fragments that express typical likes, dislikes, and current mood.

The server appends these text fragments to the primary or adjusted prompt sentence from Step 4 or Step 11, thereby creating a richer context for the generative AI model.

The server outputs an enhanced prompt sentence that encodes both objective constraints and subjective preferences, for example:
“The user prefers mild flavors and vegetable-centric dishes and avoids very spicy food. The user currently has a high stress level, so please suggest easy-to-digest meals with simple cooking steps.”

### Step 13:

The server regenerates a refined meal plan using the enhanced prompt sentence.

The server takes the enhanced prompt sentence from Step 12 as input and repeats the tokenization, tensor preparation, model inference, and decoding operations described in Steps 5 to 7.

The server performs numerical computation in the generative AI model under the new constraints and preference context, producing a refined textual meal plan that is statistically more likely to satisfy both hard constraints and soft preferences.

The server outputs an updated textual meal plan, which is then restructured and reevaluated according to Steps 8 and 9.

### Step 14:

The server transmits the finalized meal plan to the terminal for user review.

The server takes the structured meal plan object that satisfies all constraints as input and converts it into a response format suitable for the terminal.

The server may sort or group meal entries by day and meal category and may attach calculated nutritional summaries for each day.

The server outputs a response containing the structured meal plan and sends it to the terminal over the communication network.

### Step 15:

The terminal displays the meal plan and receives user approval or modification input.

The terminal takes the structured meal plan response from the server as input and renders a user interface that lists days, meals, dish names, ingredients, and nutritional summaries.

The user operates the terminal to scroll through the plan, view details, and either approve the entire plan or select dishes to modify or replace.

The terminal processes the user's actions by updating the internal representation of the meal plan and preparing an approval signal or modification instructions.

The terminal outputs a message specifying approval or modifications and transmits this message to the server.

### Step 16:

The server generates an ingredient list and matches it with sales entities.

The server takes the approved structured meal plan from Step 15 as input and aggregates ingredients across all meals, merging identical ingredients and summing their quantities.

The server consults a database of available sales entities and computes, for each entity, whether the required ingredients are available and what the estimated delivery cost and time would be.

The server performs a selection algorithm that minimizes a cost function combining distance, availability, and delivery constraints.

The server outputs a selected sales entity and a finalized ingredient list for ordering.

### Step 17:

The server constructs and transmits order data to an external information processing apparatus.

The server takes the selected sales entity and ingredient list from Step 16 as input and formats them into order data, including user address and desired delivery times.

The server performs data conversion to match the external system's expected schema and attaches authorization tokens if required.

The server outputs a network request containing the order data and sends this request to the external information processing apparatus of the selected sales entity.

### Step 18:

The server manages order status and delivery status and updates the terminal.

The server takes status messages from the external information processing apparatus as input and decodes them to update internal status records for the corresponding order.

The server maps internal status codes (such as preparing, shipped, delivered) into user-friendly messages and composes status updates.

The server outputs status update messages to the terminal at appropriate intervals or in response to terminal queries.

The terminal receives the status messages, updates the on-screen display, and allows the user to monitor the progress of ingredient preparation and delivery.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented meal planning systems mainly rely on fixed rule sets, static recipe databases, and simple filtering based on user input such as budget, number of persons, and food preferences. Such systems typically execute straightforward database lookups and do not fully leverage natural-language generative models. As a result, these systems suffer from several technical limitations when implemented on general-purpose computing hardware.

First, in existing systems, user constraints (for example, budget and preferences) are often handled as simple query parameters, and are not transformed into rich, context-aware instructions suitable for a generative AI model. This leads to inefficient use of computing resources on the model side, because the model input is either under-specified or overly generic, resulting in outputs that require extensive post-processing or user correction. The lack of a systematic mechanism to construct prompt sentences from structured user data causes redundant model invocations, increased network traffic, and unnecessary processor and memory usage.

Second, known systems generally do not provide an integrated pipeline that converts the unstructured output of a generative AI model into normalized, structured data suitable for downstream automated processing such as ingredient aggregation, cost estimation, and order generation to an online commerce system. Instead, human intervention is required to interpret generated text, manually derive a shopping list, and map ingredients to purchasable products. This manual processing increases latency, introduces errors, and prevents the system from being executed as a fully automated workflow on a server.

Third, existing solutions typically ignore real-time emotional state of the user and nutritional balance when orchestrating computations for menu generation. User feedback or mood is either treated as non-structured feedback (for example, star ratings) or is not incorporated at the system level into the control logic that drives prompt construction and model invocation. Similarly, nutritional evaluation is often left to separate tools or manual checking.

Consequently, the computing process cannot adaptively control generative model prompts according to emotional state and nutritional evaluation results, which leads to repeated trial-and-error queries to the generative AI model, wasteful recomputation, and unstable quality of generated menus.

Fourth, in conventional architectures, the linkage between generated menus and e-commerce order flows is weak or non-existent. Even when a weekly menu is automatically generated, the mapping from ingredient descriptions in natural language to normalized product identifiers, aggregation of quantities, and evaluation against user budget are typically separated processes, sometimes performed by different systems or human operators. This fragmentation leads to increased communication overhead, inconsistent data models, and failure to achieve end-to-end optimization of computational resources across menu generation, data normalization, cost evaluation, and ordering.

Thus, from a computer-technology perspective, there is a need for an improved server-side system that: (i) systematically converts structured user constraints into optimized prompt sentences; (ii) orchestrates generative AI model invocations and subsequent text parsing as a unified computational pipeline; (iii) performs automated normalization, aggregation, and cost evaluation of ingredient data; (iv) dynamically adjusts prompts and re-generation based on emotional-state analysis and nutritional evaluation; and (v) tightly integrates the above with order generation and communication with an online commerce system. Such a system should reduce the number of model calls, decrease network and processor load, and improve the overall reliability and responsiveness of a computer-implemented meal planning and ordering workflow.

The specific processing by the specific processing unit 290 of the data processing device 12 in Example 2 is realized by the following means.

The present invention provides a server comprising a processor configured to receive, via an input interface, budget information, number-of-persons information, and preference information from a terminal as structured data; to normalize the received structured data into an internal representation; to generate, based on the internal representation, a prompt sentence expressed in natural language defining menu-generation conditions and output format; to transmit the prompt sentence as inquiry data to a generative AI model and receive, from the generative AI model, text data representing menu information for a predetermined period; to parse the text data to extract dish information and ingredient information per day and structure the extracted information as per-day menu data and ingredient-list data; to normalize ingredient names contained in the ingredient-list data, integrate identical or similar ingredients, aggregate required quantities for each ingredient, and generate a bulk-purchase ingredient list; to refer, for each ingredient in the bulk-purchase ingredient list, to product information stored in product-information storage means, specify corresponding sales products, calculate purchase quantities and an estimated total cost based on unit-price information and package-unit information of the sales products, and evaluate whether the estimated total cost conforms to the budget information; to transmit the per-day menu data and the bulk-purchase ingredient list to the terminal for display and receive, from the terminal, an approval input or a modification-request input; to, when the approval input is received, generate and transmit order data including the specified sales products and the purchase quantities to a commercial-transaction system via a communication network and acquire delivery information; to obtain or estimate a user emotional state using emotion acquisition or analysis means and, based on the estimated emotional state and the per-day menu data, change menu content or configuration conditions and re-execute the generation of the prompt sentence or re-execute a query to the generative AI model so as to optimize menu proposals; and to refer, based on the menu information for the predetermined period, to nutrition-information storage means to estimate intake amounts for respective nutrients, evaluate whether a nutritional balance satisfies a predetermined condition, and, according to an evaluation result, adjust conditions included in the prompt sentence and cause the generative AI model to regenerate menus so as to obtain menu information that satisfies nutritional-balance constraints. This enables an integrated, computer-implemented workflow in which structured user constraints are efficiently converted into optimized prompt sentences, generative AI model output is automatically transformed into normalized and aggregated ingredient and menu data, emotional-state analysis and nutritional evaluation are incorporated into the control logic for prompt generation and re-generation, and menu generation is tightly coupled with automated e-commerce ordering, thereby improving computational efficiency, reducing network and processor load, and enhancing reliability and responsiveness of the meal planning and ordering system.

The term “budget information” refers to information indicating an upper limit of monetary expenditure per person, per meal, per day, or for a predetermined period, used by the system as a constraint when generating menus and estimating total cost.

The term “number-of-persons information” refers to information indicating a count of individuals for whom meals are to be planned, including, for example, a number of household members or participants to be served by the generated menus.

The term “preference information” refers to information indicating user-desired conditions related to meals, such as preferred ingredients, preferred cooking styles, dietary preferences, or ingredients and dishes to be avoided.

The term “input interface” refers to a hardware and/or software interface through which the user provides information to the system, including, for example, graphical user interfaces, web forms, mobile application screens, and associated communication modules.

The term “structured data” refers to data represented in an explicit format having predefined fields or attributes, such as records, key-value pairs, or hierarchical objects, suitable for programmatic parsing and processing by the system.

The term “internal representation” refers to a normalized data structure used within the system to represent user constraints and conditions, the internal data structure being independent of external formats and suitable for subsequent computational processing.

The term “prompt sentence” refers to a text sequence described in a natural language that encodes menu-generation conditions, constraints, and desired output formats, and that is supplied as input to a generative AI model.

The term “generative AI model” refers to a machine-learned model, such as a neural network-based language model, configured to generate text or other content in response to an input sequence including the prompt sentence.

The term “information processing platform” refers to a computing infrastructure, which may be external or internal to the server, that executes the generative AI model and processes requests from the server.

The term “text data” refers to data output by the generative AI model in a character-based form, including natural-language descriptions and optionally structured text formats, representing menu content, dish descriptions, and ingredient information.

The term “menu information” refers to information describing planned meals for a predetermined period, including, for example, days, meal times, dish names, and associated ingredients.

The term “predetermined period” refers to a time interval specified by the system or by configuration, such as one day, several days, or one week, for which menus are to be generated.

The term “per-day menu data” refers to structured data representing menus for each individual day within the predetermined period, including the dishes to be served and related information.

The term “ingredient-list data” refers to structured data representing ingredients included in the menus, including ingredient names, quantities, and units, before aggregation and normalization.

The term “normalize” refers to a process of standardizing data representations, including unifying synonymous ingredient names, formatting values into consistent units, and mapping user-facing labels to internal identifiers.

The term “bulk-purchase ingredient list” refers to a list of ingredients that are aggregated across the predetermined period, with integrated quantities, and that is suitable for use as a basis for purchasing ingredients in bulk from a commerce system.

The term “product information” refers to information associated with a purchasable item in a commerce system, including identifiers, names, unit prices, package sizes, and other attributes.

The term “product-information storage means” refers to one or more storage devices or services holding product information, such as databases, data files, or remote storage services accessible by the server.

The term “sales product” refers to a purchasable item offered by a commerce system, identified by product information and corresponding to at least one normalized ingredient.

The term “unit-price information” refers to information indicating a monetary price per package, per unit quantity, or per standardized unit of a sales product.

The term “package-unit information” refers to information indicating a packaging size or standard quantity of a sales product, used by the system to convert required ingredient quantities into purchase quantities.

The term “estimated total cost” refers to a calculated monetary value representing a sum of costs of all sales products required to satisfy the bulk-purchase ingredient list for the predetermined period.

The term “terminal device” refers to an end-user device, such as a personal computer, a smartphone, or a tablet, configured to communicate with the server and present information to the user.

The term “approval input” refers to user input indicating acceptance of generated per-day menu data and the bulk-purchase ingredient list, authorizing the system to proceed with order generation.

The term “modification-request input” refers to user input indicating a request for changes to the generated menus or ingredient lists, such as changes in dishes, ingredients, or constraints.

The term “order data” refers to structured data specifying one or more sales products, purchase quantities, and related transaction parameters to be transmitted to a commercial-transaction system.

The term “communication network” refers to one or more interconnected communication paths, such as the Internet or local area networks, over which data is exchanged between the server, terminal devices, and commerce systems.

The term “commercial-transaction system” refers to an automated system of a commerce provider configured to receive order data, process purchases, and manage delivery of goods to a user.

The term “delivery information” refers to information returned from the commercial-transaction system indicating an order status, scheduled delivery time, delivery address, and related logistics data.

The term “user emotional state” refers to an estimated condition of the user's affective or psychological state, such as stress level, mood, or satisfaction, inferred from user-related data.

The term “emotion acquisition interface” refers to an interface configured to obtain data relevant to the user emotional state, including explicit user inputs, sensor readings, or other signals.

The term “emotion analysis means” refers to hardware and/or software configured to infer the user emotional state by processing data such as operation histories, textual inputs, or biometric signals.

The term “operation-history information” refers to data representing sequences of user interactions with the user interface, such as clicks, taps, scrolling behavior, and time spent on screens.

The term “input text information” refers to textual content directly entered by the user, such as comments, free-form requests, or feedback messages.

The term “biometric information” refers to information related to physical or physiological characteristics of the user, such as heart rate, facial expressions, or voice signals, that can be used to estimate emotional state.

The term “menu content” refers to the specific dishes, ingredients, and scheduling included in generated menus.

The term “configuration conditions of menus” refers to constraints and parameters influencing menu generation, including variety, repetition limits, dietary categories, and preparation complexity.

The term “nutrition-information storage means” refers to storage resources containing nutritional data for ingredients and dishes, including nutrient types and amounts per unit quantity.

The term “intake amounts for respective nutrients” refers to estimated quantities of individual nutrients consumed over the predetermined period based on the generated menus and nutritional data.

The term “nutritional balance” refers to a relational state between intake amounts of multiple nutrients and one or more target ranges or ratios defined by nutritional guidelines.

The term “predetermined condition” refers to a threshold or set of rules applied to evaluate whether the nutritional balance is acceptable, such as minimum and maximum levels for given nutrients.

The term “constraints included in the prompt sentence” refers to specified conditions embedded in the prompt sentence that limit or guide generative AI model output, such as budget limits, excluded ingredients, variety requirements, or nutritional targets.

The term “menu proposals” refers to candidate sets of menus generated by the system, based on which the user may approve or request modifications.

The term “dynamic optimization” refers to a process of iteratively adjusting prompt sentences, constraints, and model invocations in response to feedback, emotional-state estimation, and nutritional evaluation, in order to improve subsequent menu proposals.

In one embodiment, the system includes a server, at least one terminal, and a commercial-transaction system interconnected via a communication network. The server comprises at least one processor, a main memory, a non-volatile storage device, and network interfaces. The terminal comprises a display, an input device, and a communication interface. The commercial-transaction system comprises computing resources configured to receive order data and manage product delivery.

The server executes server-side application software, for example implemented on a general-purpose operating system such as a server operating system, and may use a web application framework such as a web framework or an application framework. The server additionally accesses a database management system, for example a relational database, to store and retrieve user profiles, product information, nutritional information, and historical menu-generation data. The server further communicates with a generative AI model hosted either locally on dedicated hardware, such as a graphics processing unit, or remotely through an API of a cloud-based AI platform.

The terminal executes client-side software, such as a web browser or a native application, to present user interfaces generated by the server. The terminal displays input forms and output screens and transmits user input to the server via an encrypted communication protocol such as HTTPS.

In one embodiment, the server provides to the terminal an input interface through which the user enters budget information, number-of-persons information, and preference information. The terminal displays text fields, numeric fields, and option selectors and transmits the user inputs to the server as structured data, for example in a key-value representation. The server stores the received values in memory as an internal data structure, such as an object or a record including fields for per-person budget, household size, preferred ingredients, forbidden ingredients, dietary category, and desired variety level.

The server performs normalization of the user inputs. The server maps user-entered labels, such as “likes meat”, “no seafood”, or “vegetarian”, to internal codes using a mapping table in the database. The server converts budget information to a unified unit, for example a monetary value per person per meal, and derives additional parameters, such as a total weekly budget, by arithmetic operations on the numeric values. The server thus converts heterogeneous user inputs into a consistent internal representation used as a basis for subsequent computational processing.

The server generates a prompt sentence expressed in natural language for use with a generative AI model. The server stores in memory one or more prompt templates that specify a structure of instructions to the model, including a description of the role of the model, constraints on menu generation, a required output format, and post-processing hints. The server performs string concatenation and variable substitution to insert normalized values of the internal representation into the template. For example, the server generates a prompt sentence such as:
“Generate a 7-day dinner meal plan for 4 people. The maximum budget is 500 yen per person per meal. Consider the following preferences: likes meat. Consider the following restrictions: no seafood. List daily menus and required ingredients. Output the result in JSON-like structured text with clearly separated days and ingredients.”

In another example, the server generates a prompt sentence such as:
“Please create a one-week vegetarian dinner plan for 2 adults. The budget is 700 yen per person per meal. Prioritize high-protein, low-sugar dishes. Avoid meat, fish, and shellfish. Use tofu, beans, and vegetables as main ingredients. For each day, provide dish names, a brief description, and a list of ingredients. At the end, provide a consolidated weekly shopping list.”

In still another example, the server generates a prompt sentence such as:
“Propose a 7-day Japanese-style dinner menu for 5 people. The maximum budget is 400 yen per person per meal. Avoid all dairy products. Use commonly available ingredients in supermarkets. Make the weekly menu nutritionally balanced. For each day, list specific dish names, required ingredients with quantities, and any special seasonings.”

The server transmits the generated prompt sentence to the generative AI model. In one embodiment, the generative AI model is a transformer-based neural network language model having an encoder-decoder or decoder-only architecture with multi-head self-attention layers, feed-forward layers, layer normalization, and positional embeddings. The model parameters are stored as multi-dimensional weight matrices learned from large-scale training data. The server sends the prompt sentence as a sequence of tokens to the model via an application programming interface of an AI-serving platform. The AI-serving platform performs tokenization, converts tokens to embeddings, and executes a sequence of matrix multiplications, non-linear activations, and attention computations across multiple layers to predict subsequent tokens that form the model response.

The server receives from the generative AI model text data that represents menu information for the predetermined period. The text data may be partially structured as delimited sections per day, with lists of dish names and ingredient lists. The server stores the received text data in memory and applies a parsing algorithm to convert the text data into structured per-day menu data and ingredient-list data. The server may use rule-based parsing, such as searching for day markers (“Day 1:”, “Day 2:”), dish separators, and ingredient lines, and may additionally use natural-language processing libraries to identify ingredient phrases and quantity expressions. By using a deterministic parsing strategy tailored to the expected output format of the generative AI model, the server reduces ambiguity and standardizes the extracted data into internal data structures.

The server normalizes ingredient names contained in the ingredient-list data. The server uses a dictionary table that maps various linguistic expressions to canonical ingredient identifiers. For example, the server maps “minced beef”, “ground beef”, and “beef mince” to a single identifier representing a specific type of meat. The server also converts units (for example, grams, pieces, cups) to standardized units using predefined conversion factors stored in the database. The server thus produces normalized ingredient entries that can be reliably matched to product information stored in a product-information storage.

The server aggregates normalized ingredients across all days of the predetermined period.

The server iterates through each menu and sums numerical quantities of identical ingredients, performing arithmetic operations to generate a consolidated amount required for the entire period. The server constructs a bulk-purchase ingredient list that contains each unique normalized ingredient identifier, a total required quantity, a standard unit, and optional metadata such as quality preferences or brand preferences inferred from user settings. The server accesses product-information storage means, such as a relational database or key-value store, that maintains product catalogs of one or more commerce providers. The server queries the storage with each normalized ingredient identifier and retrieves candidate sales products that can satisfy the ingredient. The storage may contain, for each product, a product identifier, a description, a unit price, a package size, a category, and availability information. The server selects one or more sales products per ingredient, for example by applying rules that prioritize certain categories, minimize cost, or match dietary constraints. The server computes, for each selected product, a required number of packages by dividing the total required quantity by the package-unit size and performing rounding operations. The server multiplies the number of packages by the unit price and sums the results for all products, thereby calculating an estimated total cost.

The server compares the estimated total cost against the budget information derived from the user input. When the estimated total cost exceeds a threshold based on the budget, the server may mark the plan as over-budget and either generate a notification to the terminal or adjust the constraints included in a new prompt sentence to produce a more economical menu in a subsequent generation cycle. This feedback loop between budget evaluation and prompt generation improves computational efficiency because the server can guide the generative AI model toward feasible solutions, reducing the number of unproductive model queries.

The server transmits the per-day menu data and the bulk-purchase ingredient list to the terminal. The terminal renders the received data using a graphical user interface framework, displaying daily menus, dishes, and the aggregated shopping list. The user views the displayed information and operates the terminal to provide an approval input or a modification-request input. The terminal sends the user's selection back to the server as structured data.

When the server receives an approval input, the server generates order data for a commercial-transaction system. The server converts normalized ingredient identifiers and selected sales products into a list of product identifiers and purchase quantities, together with user-identifying information and delivery preferences. The server transmits the order data to the commercial-transaction system via the communication network. The commercial-transaction system processes the order and returns delivery information such as an order identifier and scheduled delivery time. The server stores the delivery information and forwards relevant details to the terminal, where the user can confirm the status and delivery schedule. In this way, the system not only performs information processing but also effects a concrete change in the physical world by controlling a flow of goods from a warehouse to the user's location. In one embodiment, the server further obtains or estimates a user emotional state and uses the emotional state to control the behavior of the generative AI model. The server can receive explicit emotional input from the user via the terminal, or can infer the emotional state by analyzing operation-history information, textual feedback, or biometric information acquired from sensors. For example, the server may infer increased stress when the user performs frequent regeneration requests or quickly abandons screens, or the server may infer a desire for comfort food when the user enters certain keywords in free-form text. The server executes an emotion analysis module that computes features such as interaction frequency, response time, textual sentiment scores, and biometric patterns, and applies a classification algorithm, such as a supervised neural network or a support vector machine, to categorize the emotional state.

The server uses the emotional-state category as an additional parameter in the internal representation. The server modifies the prompt sentence to reflect emotional-responsive constraints, such as adding phrases like “include easy-to-cook dishes suitable for busy days” or “provide light, refreshing meals to reduce heaviness.” By embedding emotional conditions in the prompt sentence, the server causes the generative AI model to shift its output distribution in a way that better matches the user's current state. Because this adjustment is implemented at the level of prompt construction and not merely as a post-processing filter, the system reduces the frequency of iterative, trial-and-error calls to the model, thereby decreasing network traffic and processor utilization on both the server and model-execution platform.

In another embodiment, the server evaluates nutritional balance based on the generated menus. The server maintains nutrition-information storage means containing nutrient data per unit quantity for ingredients and possibly for composite dishes. After parsing the text data and obtaining structured ingredients and quantities, the server calculates estimated nutrient intake for each day and for the predetermined period by multiplying ingredient quantities by nutrient values and summing across all ingredients. The server compares the nutrient intake values with predetermined conditions, such as recommended daily allowances, upper limits, or ratio ranges among energy, protein, fat, and carbohydrates. When the nutritional balance falls outside the predetermined conditions, the server adjusts the constraints in a subsequent prompt sentence, for example by adding phrases such as “increase vegetable content,” “reduce saturated fat,” or “balance protein intake across the week.” The server then re-queries the generative AI model with the adjusted prompt sentence. This closed-loop control of menu generation based on nutritional evaluation improves the precision with which the model's output satisfies complex, multi-dimensional constraints and reduces the need for manual nutritional analysis.

From a computer-technology perspective, the described configuration produces technical effects beyond mere automation of human planning. By converting structured user data into tailored prompt sentences and by using deterministic parsing and normalization pipelines, the server minimizes ambiguity and unstructured variability that would otherwise require additional model calls or manual intervention. The pipeline design, which separates prompt construction, model invocation, parsing, normalization, aggregation, cost evaluation, emotional-state adaptation, and nutritional control, allows the server to cache intermediate representations and reuse partial results when parameters change only incrementally. This reuse reduces redundant computation and network overhead. Furthermore, by encoding constraints directly into the prompt sentence instead of applying them only as a post-hoc filter, the server reduces the expected number of iterations required to obtain an acceptable menu, thereby improving throughput and latency of the overall system.

In one variation, the generative AI model is hosted locally on the same physical server or on a dedicated inference server connected via a high-speed local network. The server executes a runtime environment, such as a machine learning framework, to load model weights, perform tokenization, and manage inference. The server may exploit hardware acceleration by dispatching matrix operations to a GPU. In this configuration, the server directly controls inference batch size, beam search parameters, temperature, and maximum token length, allowing optimization of computational efficiency for the specific menu-generation task. For example, the server may select lower temperature values to produce more deterministic outputs, reducing variance and simplifying parsing, or may truncate generation at known markers to avoid unnecessary tokens, thus reducing compute cycles and memory usage. In an alternative embodiment, the system deploys multiple types of generative AI models. The server may first use a smaller, resource-efficient model to produce a coarse-grained weekly structure, and then use a larger, more capable model to refine specific days or dishes that violate constraints. The server coordinates the outputs of these models by merging structures and resolving conflicts based on rule-based logic. This hierarchical model usage improves overall computational efficiency by allocating heavyweight model usage only to parts of the menu that truly require refinement.

The system further includes variants in how product matching and cost evaluation are implemented. In one variant, the server maintains multiple product catalogs from different commerce providers and executes a selection algorithm that optimizes not only cost but also delivery time or stock availability. The server applies a scoring function that combines normalized price, shipping time, and user preference for certain vendors, then selects a combination of products that minimizes the score under budget constraints. This variant further emphasizes technical data processing and optimization within the computing infrastructure, rather than merely automating a human shopping decision.

The described embodiments illustrate that the server, through specific data structures, prompt construction algorithms, parsing and normalization procedures, emotional-state analysis, and nutritional control loops, improves the way a computing system interacts with a generative AI model and with external commerce infrastructure. The server reduces unnecessary computations, stabilizes output quality, and enables automatic generation of machine-readable lists for direct control of ordering and delivery systems. These technical advantages arise from the particular organization and transformation of data within the computing environment, rather than from business rules alone, and therefore provide an improvement to computer technology itself.

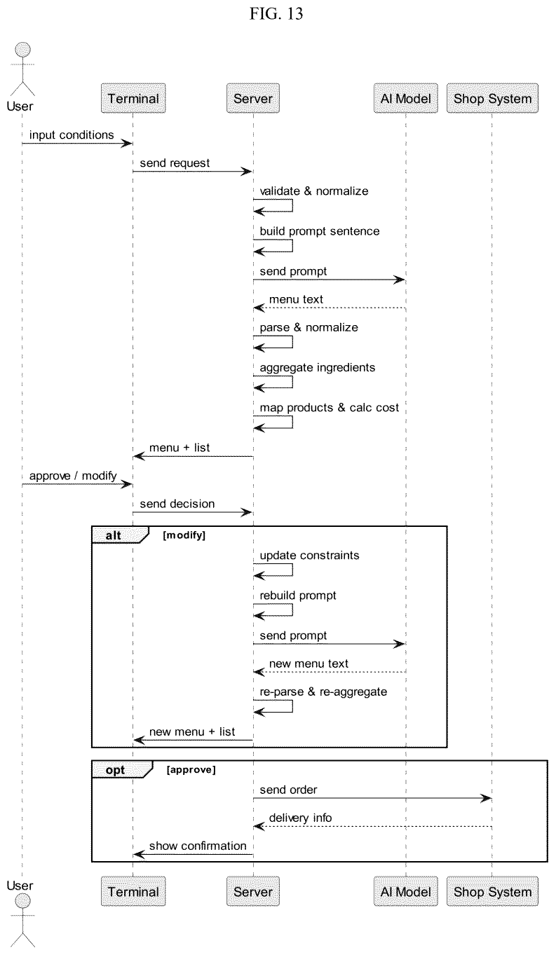

The following describes the processing flow using FIG. 13.

### Step 1:

The terminal displays an input screen for meal-planning conditions and sends user input to the server.

The terminal presents input widgets such as numeric fields for budget per person and number of persons, and selection controls for food preferences and restrictions.

The user enters values such as a per-person budget, a household size, preferred ingredients, and ingredients to be avoided, and then triggers a submit operation.

Input: raw user input values entered on the terminal UI (budget amount, number of persons, text preferences, selection flags).

Output: structured request data transmitted to the server (for example, a key-value set including budget, number_of_persons, preference_list, restriction_list).

The terminal converts the UI values into a structured payload, serializes it (for example, as a JSON object), and sends it to the server over an encrypted communication channel.

### Step 2:

The server receives and validates the structured request data.

The server parses the incoming payload, checks required fields, verifies data types (for example, numeric range of the budget, positive integer for number of persons), and confirms that preference and restriction entries conform to expected formats.

Input: structured request data from the terminal.

Output: validated and cleaned user constraint data stored in an internal data structure.

The server rejects or corrects invalid fields (for example, negative budget or missing number of persons) and logs any adjustments, thereby ensuring that downstream processing operates only on consistent input.

### Step 3:

The server normalizes the validated constraint data into an internal representation.

The server maps textual labels such as “likes meat” or “no seafood” to internal codes using lookup tables, converts budget into a unified unit (for example, per person per meal), and derives additional parameters such as total weekly budget by multiplying by the number of persons and number of days.

Input: validated user constraint data.

Output: normalized internal representation including coded preferences, coded restrictions, normalized budget values, and derived parameters.

The server performs arithmetic operations on numeric fields and table lookups on categorical fields, producing a compact, machine-friendly representation used in later steps.

### Step 4:

The server constructs a prompt sentence template and fills it with values from the internal representation.

The server selects a template corresponding to a 7-day menu scenario and inserts normalized values such as household size, per-person budget, and preference codes converted back into descriptive phrases.

Input: normalized internal representation.

Output: fully instantiated prompt sentence expressed in natural language.

The server executes string concatenation and variable substitution to produce a prompt sentence such as:
“Generate a 7-day dinner meal plan for 4 people. The maximum budget is 500 yen per person per meal. Consider the following preferences: likes meat. Consider the following restrictions: no seafood. List daily menus and required ingredients. Output the result in a clearly structured text format with separate sections for each day.”

### Step 5:

The server sends the prompt sentence to the generative AI model and receives generated text data.

The server encapsulates the prompt sentence in a request format required by the AI-serving platform, transmits it via an API call, and waits for the response.

Input: prompt sentence describing menu-generation conditions and output format.

Output: generated text data from the generative AI model representing a proposed weekly menu and associated ingredients.

The server triggers the generative AI model to perform tokenization, embedding lookup, multi-layer attention computations, and token prediction, and then captures the resulting text output from the response message.

### Step 6:

The server parses the generated text data into per-day menu data and ingredient-list data.

The server scans the text for day markers, dish names, and ingredient lines, applies rule-based or pattern-based parsing, and organizes the extracted information into structured records.

Input: free-form or semi-structured text data returned by the generative AI model.

Output: per-day menu data structures (for example, day->list of dishes) and ingredient-list data structures (for example, dish->list of ingredient entries with raw quantities).

The server uses text-processing operations such as line splitting, pattern matching, and token extraction to convert unstructured text into machine-readable data entities.

### Step 7:

The server normalizes ingredient names and units across the ingredient-list data.

The server matches ingredient strings to canonical identifiers using a dictionary table, resolves synonyms (for example, “ground beef” and “minced beef”), and converts quantities into standard units using stored conversion factors.

Input: ingredient-list data with raw ingredient names and heterogeneous units.

Output: normalized ingredient entries with unified identifiers, standardized units, and numeric quantity values.

The server performs table lookups, string similarity checks if necessary, and numeric unit conversion, thereby producing a uniform ingredient representation for aggregation.

### Step 8:

The server aggregates ingredient quantities over the entire predetermined period to build a bulk-purchase ingredient list.

The server iterates through normalized ingredient entries for all days, groups entries by canonical ingredient identifier, and sums values of standardized quantities.

Input: normalized per-dish ingredient entries for each day.

Output: bulk-purchase ingredient list containing each unique ingredient identifier and total required quantity.

The server executes arithmetic addition for grouped ingredients, generating a consolidated list that represents the full shopping requirement for the planned period.

### Step 9:

The server maps normalized ingredients to sales products and estimates total cost.

The server queries product-information storage with each ingredient identifier, retrieves candidate products with unit prices and package sizes, selects appropriate products based on internal rules, and computes the number of packages required to satisfy the total quantity.

Input: bulk-purchase ingredient list and stored product information (prices, package sizes, identifiers).

Output: product-level purchase plan including selected product identifiers, package counts, and an estimated total cost.

The server divides total ingredient quantities by package-unit sizes, applies rounding, multiplies package counts by unit prices, and sums costs across all products to compute the estimated total cost.

The server evaluates conformity of the estimated total cost with the user's budget constraints.

The server compares the estimated total cost with the derived budget threshold for the predetermined period and determines whether the plan is within budget, over budget, or near a boundary value.

Input: estimated total cost and normalized budget information.

Output: budget evaluation result (for example, within_budget flag, over_budget margin) attached to the menu plan.

The server performs comparison operations and, if the plan is over budget, can flag the plan or adjust parameters for possible regeneration in later steps.

### Step 11:

The server sends the per-day menu data and bulk-purchase ingredient list to the terminal for display.

The server assembles a response containing daily menus, dishes, normalized ingredients, aggregated quantities, and budget evaluation status, and transmits it to the terminal.

Input: structured per-day menu data, bulk-purchase ingredient list, and budget evaluation result.

Output: response payload rendered by the terminal UI as human-readable menus and shopping lists.

The server serializes the structured data, while the terminal subsequently deserializes it and converts data fields into visual components such as day-by-day lists and summary views.

### Step 12:

The user reviews the displayed menu and ingredient information and issues an approval or modification request.

The terminal renders the data received from the server, and the user examines daily menus, ingredient quantities, and budget information, then selects an action such as “Approve” or “Modify.”

Input: displayed menu and shopping list information presented on the terminal.

Output: user action captured as approval input or modification-request input and sent back to the server.

The terminal encodes the user action together with a plan identifier and transmits it to the server as structured control data.

### Step 13:

The server processes a modification-request input by altering constraints and regenerating menus, if requested.

The server interprets the modification request (for example, remove certain dishes, adjust budget, or change preferences), updates the internal representation accordingly, reconstructs a new prompt sentence with revised constraints, and re-invokes the generative AI model.

Input: modification-request input and the current internal representation of constraints and menu data.

Output: updated menu plan generated by repeating the sequence of prompt generation, model invocation, parsing, normalization, aggregation, and cost evaluation.

The server executes data updates on the internal representation, produces an adjusted prompt sentence, and re-runs the generative AI model and subsequent processing pipeline, then returns the revised plan to the terminal.

### Step 14:

The server processes an approval input by generating and transmitting order data to the commercial-transaction system.

The server extracts the final bulk-purchase ingredient list and associated product-level purchase plan, composes order data including product identifiers, package counts, delivery address, and desired delivery slot, and sends this data to the commercial-transaction system.

Input: approval input from the user and the finalized product-level purchase plan with user profile information.

Output: order data sent to the commercial-transaction system and received delivery information (such as order ID and delivery schedule).

The server formats the order data according to the commerce system's schema, transmits it via a network API, receives a confirmation response, and attaches the returned delivery information to the user's plan record.

### Step 15:

The server obtains and uses user emotional-state information to adapt prompt generation and menu proposals.

The server collects emotional indicators, such as explicit mood selections, textual feedback, or interaction-pattern features, and applies an emotion analysis module that classifies the user's emotional state into categories.

Input: emotional indicators derived from user interactions and optional sensor data.

Output: emotional-state parameter stored in the internal representation and used to modify future prompt sentences.

The server updates the prompt sentence by adding or modifying constraints that reflect the emotional state (for example, easier recipes when stress is high), and, when regenerating menus, the server passes the modified prompt sentence to the generative AI model, thereby producing menu proposals more aligned with the user's current condition.

### Step 16:

The server evaluates nutritional balance and adjusts prompt conditions for subsequent generations.

The server uses nutritional information for each ingredient to compute daily and weekly nutrient intake based on the generated menus, compares these values with predetermined nutritional criteria, and determines whether the menus satisfy required nutritional balance.

Input: structured menu and ingredient data combined with nutrient-per-unit information from nutrition-information storage.

Output: nutritional evaluation results and adjusted prompt constraints for potential re-generation.

The server multiplies ingredient quantities by nutrient values, sums nutrients by day and by week, and uses comparisons against target ranges to identify deficiencies or excesses. When imbalances are detected, the server modifies the prompt sentence conditions (for example, request more vegetables or reduce fat), and on the next call to the generative AI model, the updated prompt sentence guides the model toward nutritionally balanced outputs.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional meal-planning and ingredient-ordering systems mainly treat menus as static content generated from simple rule-based filtering of recipes by budget, number of persons, and food preference. Such systems typically send fixed queries to recipe databases or template engines, and then separately generate shopping lists and orders. As a result, several technical problems arise in terms of computer technology itself.

First, in conventional systems, generation of meal plans and generation of ingredient orders are implemented as loosely coupled, manually configured processes. A server often receives heterogeneous user inputs (numeric budgets, natural language preferences, and unstructured comments) and passes them directly to disjoint modules, thereby requiring complex, ad hoc glue code. Because there is no unified representation of user conditions, including emotion, the server cannot efficiently orchestrate downstream computations such as menu creation, nutrition evaluation, and ordering. This leads to redundant parsing, repeated data conversions, and increased processing latency.

Second, existing systems do not systematically leverage generative artificial intelligence models via a well-structured prompt generation mechanism. In many conventional approaches, a generative model, if used at all, is invoked with manually written prompts or static templates. This prevents dynamic adaptation of prompts to changing user conditions, including emotion or nutrition balance feedback, and causes the server to perform multiple heuristic post-processing steps to repair the generated output. Consequently, computational resources are wasted reconciling inconsistent menu formats, and the overall throughput and reliability of the system are degraded.

Third, conventional architectures generally do not integrate emotion recognition and nutrition evaluation into a closed-loop control flow for the generative model. Emotion recognition, if present, is treated as a separate analytics function, and nutrition evaluation is often executed as an offline or batch process. These fragmented components do not feed back into prompt generation in a structured manner, so the server cannot automatically regenerate or adjust meal plans based on both emotional state and quantitative nutrition evaluation. This lack of feedback leads to additional manual interventions, repeated calls to external services, and difficulty in scaling the system while maintaining responsiveness.

Fourth, existing systems typically lack an integrated mechanism for transforming generated meal plans into a normalized ingredient list, mapping that list to purchasable merchandise information from external commerce platforms, and then coordinating order submission and delivery tracking in an automated manner. The absence of such an end-to-end orchestration layer forces the server to maintain multiple, incompatible data formats and to execute many separate request-response cycles with external platforms. This increases the complexity of scheduling, error handling, and status management, and can result in inconsistent or failed orders when conditions change or partial data become invalid.

Accordingly, there is a need for an improved computer-implemented system that: (i) unifies heterogeneous user inputs, including emotion-related information, into structured condition information; (ii) automatically generates and regenerates prompt sentences for a generative artificial intelligence model in response to emotion and nutrition feedback; (iii) performs structured parsing, ingredient aggregation, and nutrition evaluation in a coordinated pipeline; and (iv) transforms adjusted meal plan information directly into merchandise selection, ordering, and delivery control through external electronic commerce and logistics apparatuses. By solving these issues at the system and data-flow level, the invention aims to improve the technical functioning of the server, reduce redundant computation, enhance robustness of the end-to-end meal planning and ordering process, and provide lower latency and more reliable operation under varying user conditions.

The specific processing by the specific processing unit 290 of the data processing device 12 in Application Example 2 is realized by the following means.

The present invention provides a server comprising a processor configured to acquire, via an input interface, information including a user budget, a number of persons, a food preference, and information related to a user emotion; to normalize numerical information and character information included in the acquired information and convert the numerical information and the character information into a predetermined format; to specify an emotional state of the user based on the acquired character information and the information related to the user emotion; to automatically generate, from condition information including the user budget, the number of persons, the food preference, and the emotional state of the user, a prompt sentence for instructing a generative artificial intelligence model to generate a meal plan; to input the prompt sentence into the generative artificial intelligence model and cause the generative artificial intelligence model to perform character-string computation to generate meal plan information for a predetermined period; to analyze the generated meal plan information, extract ingredient information necessary for each meal over the predetermined period, and generate an ingredient list in which quantities of duplicate ingredients are aggregated and integrated; to refer, for each ingredient included in the ingredient list, to a nutrition information storage unit to acquire nutrition values, perform predetermined aggregation computation to evaluate a nutrition balance of the meal plan information as a whole, and regenerate or correct the meal plan information in accordance with an evaluation result; to reconstruct the prompt sentence based on the emotional state of the user and the evaluation result of the nutrition balance, reinput the reconstructed prompt sentence into the generative artificial intelligence model, and adjust the meal plan information so as to be adapted to the emotional state of the user and the nutrition balance; to finalize the ingredient list based on the adjusted meal plan information, to control an information provision function of an external merchandise information provision apparatus so as to acquire merchandise information corresponding to the ingredient list, and to select merchandise candidates from the acquired merchandise information; to transmit, in accordance with a predetermined communication procedure and based on the merchandise candidates and residence information of the user, order information for ingredients and delivery destination information to a nearest electronic commerce apparatus, and to acquire information related to a delivery status; and to transmit the meal plan information, the ingredient list, information related to the nutrition balance, and the information related to the delivery status to a user terminal and cause the user terminal to present the transmitted information to the user. This enables a unified, feedback-driven computation pipeline in which the server automatically transforms heterogeneous user inputs into structured condition information, dynamically generates and regenerates prompt sentences for the generative artificial intelligence model based on emotion and nutrition evaluation, produces consistent meal plan information and aggregated ingredient lists, and directly controls merchandise selection, ordering, and delivery through external platforms, thereby improving internal data flow efficiency, reducing redundant processing, enhancing robustness of the overall system, and providing faster and more reliable end-to-end meal planning and ingredient procurement.

The term “user budget” refers to monetary information indicating an amount of expenditure that a user allows for purchasing ingredients or meals for a predetermined period or per meal, which is used as a constraint in generating a meal plan.

The term “number of persons” refers to numerical information indicating how many individuals are intended to be served by the meal plan, and is used to determine portions, ingredient quantities, and overall scale of the meals.

The term “food preference” refers to information indicating user-specific conditions regarding desired or undesired foods, including liked ingredients, disliked ingredients, dietary restrictions, and preferred culinary styles.

The term “information related to a user emotion” refers to data from which an emotional state of a user can be inferred, including, for example, natural language text, voice signals, images, or explicit user selections indicating mood or feelings.

The term “input interface” refers to a logical and physical interface configured to receive information from a user or a user terminal, including graphical user interfaces, programmatic interfaces, or communication endpoints that accept the user budget, number of persons, food preferences, and emotion-related information.

The term “normalize” refers to processing in which raw numerical and character information is converted into a standardized internal representation, including operations such as parsing, unit conversion, formatting, tokenization, and removal of inconsistencies in the data.

The term “emotional state” refers to a classified psychological condition of a user, such as joy, sadness, fatigue, excitement, or calmness, that is derived from emotion-related information and used as a parameter in generating or adjusting a meal plan.

The term “condition information” refers to structured data that aggregates the user budget, number of persons, food preferences, emotional state, and optionally additional constraints, and that serves as an input to generation of a prompt sentence.

The term “prompt sentence” refers to a machine-interpretable natural language instruction generated from condition information, which is provided to a generative artificial intelligence model to request generation or adjustment of meal plan information.

The term “generative artificial intelligence model” refers to a computational model configured to generate new content, including text, based on input prompts, using techniques such as deep learning, probabilistic modeling, or neural network architectures.

The term “character-string computation” refers to internal processing by a generative artificial intelligence model in which textual tokens are analyzed and generated in sequence or parallel to produce output text based on an input prompt.

The term “meal plan information” refers to data representing one or more proposed meals for a predetermined period, including, for example, dish names, meal times, and descriptive information associated with each meal.

The term “predetermined period” refers to a time interval for which a meal plan is generated, such as a day, several days, or a week, which may be set by the system or specified by the user.

The term “ingredient information” refers to data describing individual food components needed to prepare a meal, including ingredient names, quantities, and optionally units and preparation notes.

The term “ingredient list” refers to an aggregated collection of ingredient information derived from one or more meals in the meal plan, in which duplicate ingredients are combined and total quantities are calculated.

The term “nutrition information storage unit” refers to a data storage component configured to store nutritional values for ingredients or foods, including calories, macronutrients, and micronutrients, and to provide such values in response to queries.

The term “nutrition balance” refers to an assessment of how well a meal plan meets target nutritional criteria, based on aggregated nutritional values such as energy, macronutrients, and micronutrients over the predetermined period.

The term “evaluation result” refers to data produced by computation that assesses the nutrition balance or other quality metrics of the meal plan information, and that is used to determine whether regeneration or correction of the meal plan is required.

The term “reconstruct the prompt sentence” refers to processing in which an existing prompt sentence is modified or regenerated by incorporating updated information, such as an emotional state or a nutrition evaluation result, in order to request an adjusted meal plan from the generative artificial intelligence model.

The term “adjust the meal plan information” refers to modifying previously generated meal plan information, by changing, replacing, or reordering meals or dishes, so as to better satisfy conditions including emotional state and nutrition balance.

The term “external merchandise information provision apparatus” refers to an external system or service configured to provide information about purchasable items, such as food products, including item descriptions, identifiers, prices, and availability.

The term “merchandise information” refers to data describing purchasable items corresponding to ingredients, including, for example, product names, package sizes, prices, and inventory information.

The term “merchandise candidates” refers to one or more selected purchasable items deemed suitable to satisfy the ingredient list, chosen from merchandise information obtained from an external merchandise information provision apparatus.

The term “electronic commerce apparatus” refers to an external system configured to receive orders for goods via a communication network, process payments, and manage at least part of the logistics or fulfillment operations.

The term “order information” refers to data specifying a request to purchase items, including item identifiers, quantities, prices, and any necessary transaction details for submitting an order to an electronic commerce apparatus.

The term “delivery destination information” refers to data specifying a physical location to which ordered items are to be delivered, such as a user residence address and optionally preferred delivery time windows.

The term “delivery status” refers to information indicating a state of processing and physical movement of ordered items, including, for example, order accepted, preparing, shipped, out for delivery, or delivered.

The term “user terminal” refers to an electronic device operated by a user, such as a mobile device, a tablet device, or a general-purpose computing device, that is configured to transmit input information to the server and present output information from the server to the user.

The term “present the transmitted information” refers to causing a user terminal to display, play back, or otherwise output meal plan information, ingredient lists, nutrition balance information, and delivery status information to the user in a perceivable form.

In one embodiment, a system for generating and executing a personalized meal plan is implemented as a distributed computer system including at least one server and a plurality of terminals. The server includes at least one processor, a main memory, a non-volatile storage device, and one or more network interfaces. The terminal includes a processor, a display, an input device, a network interface, and optionally a camera and a microphone. The terminal is, for example, a mobile computing device, a tablet computing device, or a general-purpose computing device. The server is, for example, a rack-mounted computing device deployed in a data center or a virtual machine instantiated on a cloud computing infrastructure. The server executes a server program implemented using a programming language such as a general-purpose high-level language and uses a web application framework such as a general-purpose web framework (for example, a framework capable of handling HTTP requests and routing, comparable to Flask or FastAPI). The terminal executes a client program implemented, for example, with a cross-platform user interface framework comparable to React Native, or a browser-based web application.

The server stores, in the non-volatile storage device, a plurality of software modules including: an input management module, a normalization module, an emotion recognition module, a prompt generation module, a generative AI client module, a menu parsing module, an ingredient aggregation module, a nutrition evaluation module, a menu adjustment module, a merchandise mapping module, an ordering control module, and a delivery status management module. The server further stores one or more data repositories including: a user profile repository, a menu repository, an ingredient repository, and a nutrition information repository. Each repository is implemented using a relational database management system, such as a SQL database, and stores data in structured tables with defined schemas.

The terminal executes a user interface module that displays form elements for receiving information including a user budget, a number of persons, and a food preference. The terminal also displays user interface elements for receiving mood-related comments as natural language text and, when permitted by the user, for activating the camera and microphone to capture facial images and voice signals. The terminal transmits the acquired information to the server using a structured data format such as JSON over a secure communication protocol such as HTTPS.

The server receives the JSON data and stores the raw data in the user profile repository under a unique request identifier. The server then executes the normalization module to convert raw budget text into a numeric value in a canonical currency unit, to standardize the number of persons as an integer, and to tokenize the food preference and mood comments into lexical tokens. The normalization module uses a natural language processing library such as an NLP toolkit (for example, comparable to NLTK or spaCy) to perform tokenization, part-of-speech tagging, and named entity recognition to identify ingredient names, cuisine types, and explicit restrictions such as “no seafood” or “low carb.”

The server executes the emotion recognition module to determine an emotional state. In one embodiment, the emotion recognition module includes a neural network classifier trained using a deep learning framework comparable to TensorFlow or another machine learning framework. The neural network has, for example, a multi-layer architecture including an input layer that receives text embeddings, one or more hidden layers configured as bidirectional recurrent layers or transformer-based self-attention layers, and an output layer that outputs probabilities over a fixed set of emotion classes such as joy, sadness, fatigue, excitement, and calmness. The server converts the text tokens into dense vector representations using a pre-trained word-embedding or sentence-embedding model and inputs the embeddings into the neural network. The neural network is trained in advance on labeled text data using a cross-entropy loss function and an optimization algorithm such as stochastic gradient descent or an adaptive gradient method. During training, the server updates the network weights according to the gradient of the loss with respect to each weight, using backpropagation. Data augmentation techniques such as synonym replacement and random deletion of non-critical tokens may be applied to increase robustness.

In another embodiment, the emotion recognition module additionally or alternatively processes facial images and voice signals. The server uses a computer vision library comparable to OpenCV to detect faces in an image, align the faces, and extract facial landmarks. The server inputs the aligned facial images into a convolutional neural network having multiple convolutional and pooling layers, followed by fully connected layers and a softmax output layer that produces emotion probabilities. The server also computes audio features, such as Mel-frequency cepstral coefficients, pitch contours, and energy, from the voice signal and inputs these features into a recurrent neural network or a temporal convolutional network that outputs emotion probabilities. The server fuses text-based, image-based, and audio-based emotion probabilities using a weighted average or a learned fusion network to obtain a final emotional state for the request.

The server represents the user budget, number of persons, normalized food preferences, and the emotional state as a structured condition record. The condition record is stored as a JSON object or as multiple rows in a normalized relational schema. The prompt generation module converts the condition record into a prompt sentence. The prompt generation module uses a rule-based template engine to map condition fields into natural language. For example, if the budget is 500 units of currency per person, the number of persons is 4, the food preference is “meat dishes, no seafood,” and the emotional state is joy, the prompt generation module generates a prompt sentence such as:
“Please propose a one-week dinner menu for 4 people, with a budget of 500 per person per meal. The family prefers meat dishes and wants to avoid seafood. The family is in a joyful mood, so make the menu slightly more festive and luxurious.”

The server then uses the generative AI client module to send the prompt sentence to an external generative AI model. The generative AI model is, for example, a large-scale neural network configured as a transformer architecture having multiple self-attention layers, feed-forward layers, and layer normalization operations. The generative AI model is pre-trained on a large corpus of text and is fine-tuned for instruction-following behavior. The server transmits the prompt sentence to an inference service that executes the generative AI model, specifying parameters such as a maximum number of output tokens and a temperature that controls randomness.

The generative AI model performs token-level prediction using internal character-string computations, in which each token is embedded, passed through successive self-attention layers that compute attention scores and weighted combinations of token representations, and processed by position-wise feed-forward networks. At each decoding step, the model computes a probability distribution over the vocabulary and selects a next token based on the distribution. The model repeats this process until an end-of-sequence token is produced or a token limit is reached. The inference service returns the generated text to the server.

The server executes the menu parsing module to convert the generated text into structured meal plan information. The server may, for example, instruct the generative AI model through the prompt sentence to output each day's meals in a recognizable pattern such as “Monday: [dish list]”. The menu parsing module splits the text into lines, identifies day labels, and extracts dish names and optional descriptions. The server stores the parsed meal plan information in the menu repository. If the generated text is not in the desired structure, the server may generate a secondary prompt sentence such as:
“Reformat the following weekly menu into a JSON-like list of days and dishes: [menu text].” and send the secondary prompt to the generative AI model to obtain a more structured representation.

The server executes the ingredient aggregation module to derive an ingredient list from the meal plan information. The server queries the ingredient repository, which stores mappings from dish names to ingredient records. Each ingredient record includes an ingredient name, a standard unit, and a quantity for a base number of persons. The server scales each quantity by the number of persons in the condition record and aggregates identical ingredients across all dishes and days. The server uses deterministic rules to combine quantities and to convert different units into a common unit where possible. For example, if one dish requires 100 grams of onions and another dish requires 1 onion, the server uses a conversion factor from the ingredient repository to compute a combined quantity.

The server executes the nutrition evaluation module to evaluate a nutrition balance. The nutrition information repository stores, for each ingredient, nutritional values such as energy, protein, fat, carbohydrate, fiber, vitamins, and minerals. The server retrieves these values for each ingredient in the ingredient list and performs numeric aggregation using a numerical computation library such as a matrix or array processing library. The server computes totals per day and per predetermined period and compares them with target ranges derived from nutritional guidelines. The nutrition evaluation module outputs an evaluation result indicating, for example, that the total fat is above a threshold or that the fiber content is below a threshold.

The server executes the menu adjustment module to adjust the meal plan based on the emotional state and the evaluation result. The server modifies the condition record to include parameters such as “reduce total fat,” “increase vegetables,” or “easy-to-prepare dishes” and reconstructs a prompt sentence. For example, if the emotional state is fatigue and the evaluation result indicates excessive complexity of preparation, the prompt generation module may construct a new prompt sentence such as:
“The user is feeling tired today. Based on the following weekly menu, please adjust the dishes to be easier to prepare and more comforting, while keeping the budget of 500 per person and avoiding seafood. Current menu: [menu text].”

The server sends the reconstructed prompt sentence to the generative AI model, receives an adjusted menu, and re-runs the menu parsing, ingredient aggregation, and nutrition evaluation modules. The server may perform multiple iterations until the menu satisfies predetermined nutrition thresholds and is consistent with the emotional state.

The server executes the merchandise mapping module to transform the finalized ingredient list into merchandise candidates. The server communicates with an external merchandise information provision apparatus via an application programming interface to obtain data for items that match ingredient names. The server applies matching rules that map ingredient names to product categories and uses filters such as package size, price, and availability. The server selects one or more merchandise candidates for each ingredient using scoring rules that balance cost and total number of items, for example by preferring packages that minimize leftover quantities while meeting the required amount.

The server executes the ordering control module to construct order information and delivery destination information. The server formats the order information according to the protocol required by an electronic commerce apparatus and includes item identifiers, quantities, user identification, and billing information. The server transmits the order information and delivery destination information to the electronic commerce apparatus using a predetermined communication procedure. The electronic commerce apparatus returns an order confirmation and delivery information that include an estimated delivery date and a tracking identifier. The server records this information in the delivery status management module and periodically queries the electronic commerce apparatus or a logistics provider to update delivery status.

The server sends updated status to the terminal, which displays a tracking interface.

The described architecture and data flow improve computer technology beyond a mere automation of human meal planning. By representing user budget, number of persons, food preferences, and emotional state as a structured condition record and by generating prompt sentences programmatically, the server reduces redundant parsing and conversion operations that would otherwise be necessary for each subsystem. Because the server uses a modular pipeline with specific data structures and neural models at distinct stages, it can reuse intermediate representations and minimize data duplication, thereby reducing memory bandwidth and improving cache utilization during processing.

The emotion recognition module and the nutrition evaluation module form a feedback loop with the prompt generation module and the generative AI client module. This closed-loop design allows the server to converge on a menu satisfying multiple quantitative and qualitative constraints using a small number of generative AI model calls, rather than relying on trial-and-error manual editing or ad hoc repeated calls. As a result, the server reduces the total number of network round trips and the amount of data transmitted to the generative AI model service, thereby reducing communication load and latency.

The use of explicit neural network architectures and training methods for emotion recognition allows the server to classify emotions with higher accuracy than conventional rule-based sentiment analysis. The trained models operate on numerical feature vectors and learn non-linear relationships between linguistic, visual, and acoustic cues and emotional categories. This non-linear mapping cannot be easily replicated by simple deterministic rules or human heuristics. Because the emotional state directly influences prompt construction, the improvement in emotion classification accuracy leads to more appropriate and precise adjustments of the generated meal plan, thereby reducing the number of regeneration iterations and stabilizing system behavior.

The generative AI model used for menu generation is invoked using systematically generated prompt sentences that embed structured condition information. This approach differs from manual prompt engineering in that the server uses a deterministic mapping from structured fields to language segments, ensuring consistency across requests and enabling automated post-processing. The server can therefore predict and enforce the format of the generated text, allowing efficient parsing into data structures and reducing errors that would otherwise occur if free-form text were used. This controlled prompting strategy constitutes a non-conventional use of generative models that improves computational reliability and downstream processing efficiency.

In addition, the integration of ingredient aggregation, nutrition evaluation, and merchandise mapping into a single coordinated pipeline allows the server to maintain a canonical ingredient list that is shared by all modules. This design reduces the number of intermediate representations and conversion steps between JSON, database records, and external API formats. By reducing the complexity and multiplicity of data transformations, the system decreases the probability of inconsistency and lowers processing time.

The described system is not limited to a single implementation. In another embodiment, the server executes a different type of emotion recognition model using a support vector machine or a gradient-boosting model trained on engineered features extracted from text, images, and audio. In yet another embodiment, the generative AI model is an in-house transformer model running on a graphics processing unit cluster managed by the server, rather than an external inference service. In this case, the server deploys a model parallelism or data parallelism strategy to distribute computation across multiple processing units and uses a model quantization technique to reduce memory usage and increase inference speed.

In an alternative embodiment, the nutrition evaluation module incorporates additional constraints such as glycemic index or specific micronutrient thresholds. The server can adjust the weight of each constraint when reconstructing the prompt sentence, expressing preferences like “prioritize low glycemic index meals even if variety is reduced.” The generative AI model, guided by such constraints, generates menus that would be difficult to compose manually due to the complexity of multi-dimensional nutrient optimization.

In another embodiment, the merchandise mapping module is configured to interact with multiple electronic commerce apparatuses and to perform load balancing based on current system response times and network latency. The server may choose a nearest or fastest supplier based on real-time performance metrics, thereby improving overall end-to-end delivery times. The server can also cache commonly used mapping results between ingredients and merchandise candidates to reduce the number of external API calls and further decrease communication overhead.

The system therefore operates as an improved technical platform for end-to-end meal planning and ingredient procurement. The server coordinates multiple specialized modules, each performing specific data processing and numerical computation tasks, and integrates these into a coherent pipeline that takes heterogeneous user inputs, uses learned non-linear models to infer emotion, constructs precise prompt sentences for a generative AI model, transforms generated text into structured data, performs nutrition analysis, and controls ordering and delivery operations through external devices. This integrated design yields improvements in processing speed, accuracy of meal and emotion alignment, reliability of ordering workflows, and efficiency of data management, and thus constitutes an improvement in computer technology.

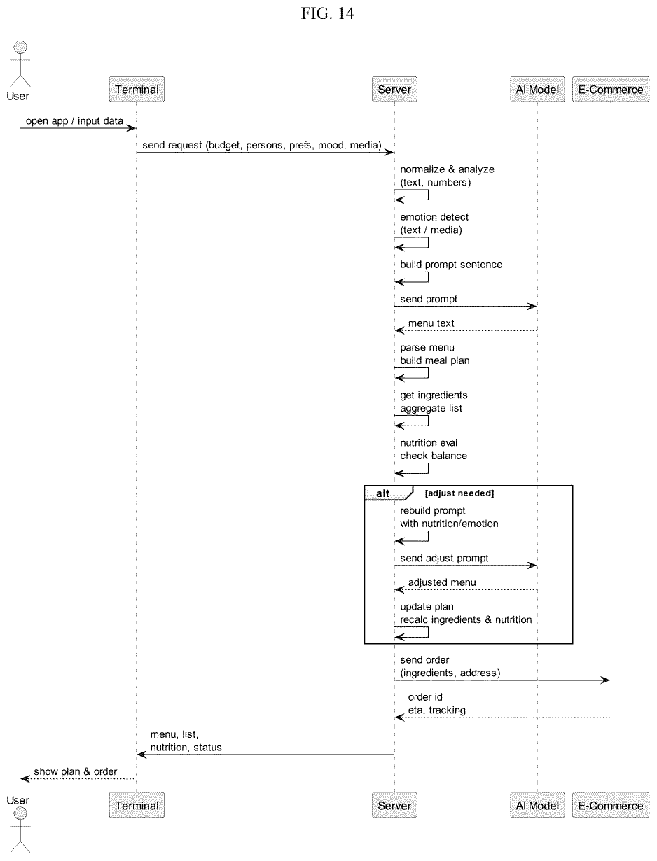

The following describes the processing flow using FIG. 14.

### Step 1:

User operates the terminal to start an application for meal planning and ingredient ordering.

Input: No prior data; user simply launches the application.

Output: Terminal displays an initial screen with input fields for budget, number of persons, food preferences, and emotion-related information.

Terminal loads a user interface layout from local resources or from the server, initializes form controls, and prepares data structures to hold user input.

### Step 2:

User enters meal-related conditions into the terminal.

Input: Visual form fields (text boxes, drop-downs, sliders) presented on the terminal.

Output: Raw input values such as strings for budget (e.g., “500 per person”), an integer or string for number of persons (e.g., “4”), a text description of food preferences (e.g., “meat dishes, no seafood”), and optional mood comments (e.g., “We are in a good mood and want something special this week”).

User fills each field and optionally types a free-text comment describing mood or context.

### Step 3:

Terminal optionally acquires emotion-related media data.

Input: User consent to use camera and microphone, and activation of corresponding UI controls.

Output: One or more image frames of the user's face and one or more audio segments containing user speech or vocal sounds.

Terminal activates a camera module and a microphone module, captures frames and short audio clips, compresses them to standard formats (e.g., JPEG for images, a compressed audio codec for sound), and stores them temporarily in memory.

### Step 4:

Terminal constructs and sends a structured request to the server.

Input: Raw textual input (budget, number of persons, food preferences, mood comments) and optional media files (images and audio).

Output: A structured request message encoded as JSON containing the textual fields and references or binary payloads for the media, transmitted to the server via HTTPS.

Terminal serializes user input into a JSON object with keys such as “budget,” “persons,” “preferences,” and “mood_text,” attaches media files as multipart fields or encoded strings, and sends an HTTP POST request to a predefined server endpoint.

### Step 5:

Server receives and stores the raw request data.

Input: HTTP request containing JSON and optional media payloads from the terminal.

Output: A stored request record including a unique request identifier and raw input values in a database.

Server parses the HTTP request body, validates required fields, assigns a unique identifier, and inserts the raw fields and media references into tables or documents in persistent storage for subsequent processing.

### Step 6:

Server normalizes numeric and textual fields.

Input: Raw budget string, number-of-persons string or numeric, and food preference/mood comment strings from the stored request.

Output: A normalized condition record including numeric budget in a standard currency unit, an integer number of persons, cleaned preference tokens, and cleaned mood tokens.

Server parses the budget string to extract numeric values and units, converts currency expressions into a canonical form, casts number-of-persons into an integer, tokenizes textual fields using a natural language processing library, lowercases tokens, removes extraneous punctuation, and flags recognized dietary terms (e.g., “no seafood,” “low carb”).

### Step 7:

Server performs text-based emotion recognition.

Input: Normalized mood comment tokens (and optionally preference tokens) taken from the condition record.

Output: A probability distribution over emotion classes and a selected primary emotional state (e.g., “joy”).

Server converts the tokens into embedding vectors using a pre-trained text embedding model, feeds the vectors into a trained neural network classifier, performs matrix multiplications and non-linear activation functions through the network layers, and computes a softmax output to obtain probabilities for each emotion category, selecting the highest probability as the primary emotional state.

### Step 8:

Server performs image-based and audio-based emotion recognition (if media are available).

Input: Captured facial images and audio clips associated with the request.

Output: Additional emotion probability distributions from facial analysis and voice analysis, and possibly intermediate feature vectors.

Server uses a vision library to detect faces and align them, inputs face images into a convolutional neural network, propagates pixel values through convolution and pooling layers to obtain a predicted emotion distribution; for audio, server computes time-frequency features (e.g., Mel-frequency coefficients), inputs these features into a temporal neural model, and computes another emotion distribution.

### Step 9:

Server fuses multiple emotion signals into a final emotional state.

Input: Text-based emotion distribution, image-based emotion distribution, and audio-based emotion distribution (where available).

Output: A fused emotion probability distribution and a final emotional state label used in subsequent processing.

Server combines probability vectors using a weighted averaging rule or a learned fusion mapping, normalizes the combined vector so that probabilities sum to one, and selects the label of the highest-probability emotion as the final emotional state (e.g., “excitement” or “fatigue”).

Server constructs a structured condition object.

Input: Normalized budget value, normalized number of persons, processed food preference tokens, and final emotional state label.

Output: A structured condition object stored as JSON or as a group of related database records that encapsulate all constraints and context for the meal plan.

Server assigns fields such as “budget_per_person,” “persons,” “preference_tags,” and “emotion_label,” and stores this object linked to the request identifier for retrieval by other modules.

### Step 11:

Server generates a primary prompt sentence for the generative AI model.

Input: Structured condition object including budget, number of persons, preference tags, and emotion label.

Output: A natural-language prompt sentence describing the requirements for the meal plan.

Server passes the condition fields to a template engine, selects phrase variants depending on the emotion (e.g., “festive,” “comforting,” “easy to prepare”), concatenates predetermined fragments with inserted numeric values, and produces a prompt sentence such as:
“Please propose a one-week dinner menu for 4 people, with a budget of 500 per person per meal. The family prefers meat dishes and wants to avoid seafood. The family is in a joyful mood, so make the menu slightly more festive and luxurious.”

### Step 12:

Server requests menu generation from the generative AI model.

Input: Primary prompt sentence describing constraints, preferences, and emotional context.

Output: A generated textual menu description for a predetermined period (e.g., one week).

Server transmits the prompt sentence as part of a request payload to a generative AI inference service, specifies model parameters such as maximum tokens and sampling temperature, and waits for the response containing the model's generated text.

### Step 13:

Server parses the generated menu text into structured meal plan information.

Input: Generated menu text returned by the generative AI model.

Output: A structured meal plan object, for example a mapping from day names to lists of dish entries.

Server splits the text into lines, detects day labels such as “Monday”, extracts dish titles and descriptions using pattern matching or regular expressions, constructs an internal list of days with associated dishes, and stores this structure in the meal plan repository linked to the request.

### Step 14:

Server derives a preliminary ingredient list from the meal plan.

Input: Structured meal plan object containing days and dishes.

Output: A preliminary ingredient list detailing ingredient names and quantities per dish before aggregation.

Server looks up each dish in an ingredient repository, retrieves base ingredient records including name, unit, and base quantity per person, multiplies each quantity by the number of persons in the condition object, and compiles all ingredient records for all dishes into a single preliminary list.

### Step 15:

Server aggregates duplicate ingredients and standardizes quantities.

Input: Preliminary ingredient list with potentially repeated ingredient names and heterogeneous units.

Output: A consolidated ingredient list in which each ingredient appears once with a total standardized quantity.

Server groups ingredient records by normalized ingredient name, converts each quantity into a standard unit using predetermined conversion factors (e.g., items to grams), sums quantities within each group, and outputs a list of unique ingredients with aggregated quantities and selected standard units.

### Step 16:

Server evaluates nutritional balance of the meal plan.

Input: Consolidated ingredient list and nutrition information from the nutrition repository for each ingredient.

Output: Daily and overall nutritional totals, and a nutrition evaluation result that indicates whether targets are met or violated.

Server queries the nutrition repository to obtain nutrient vectors for each ingredient (calories, protein, fat, carbohydrates, etc.), multiplies each nutrient vector by the ingredient quantity factor, sums nutrient vectors over all ingredients for each day and for the entire period, and compares these sums with target ranges; server then flags any metrics that exceed or fall below thresholds and stores an evaluation summary.

### Step 17:

Server determines whether menu adjustment is necessary.

Input: Final emotional state, nutrition evaluation result, and possibly metadata about dish complexity (e.g., number of steps per recipe).

Output: A decision flag indicating whether the current menu is acceptable or needs adjustment, and, if needed, a set of adjustment directives (e.g., “reduce fat,” “simplify preparation”).

Server runs rule-based logic or a small decision model that considers emotion (e.g., “fatigue” →need easy recipes) and evaluation flags (e.g., high fat→reduce fatty dishes), generates a list of required changes, and sets a boolean or multi-valued status for subsequent steps.

### Step 18:

Server reconstructs a secondary prompt sentence for menu adjustment when necessary.

Input: Adjustment directives, original condition object, and existing meal plan text.

Output: A secondary prompt sentence instructing the generative AI model how to modify or regenerate the menu.

Server embeds the directives in the prompt, such as:
“The user is feeling tired today. Based on the following weekly menu, please adjust the dishes to be easier to prepare and more comforting, while keeping the budget of 500 per person and avoiding seafood. Current menu: [menu text].”

Server concatenates the original menu as context and reformulates the request so that the generative AI model outputs a revised menu aligned with the directives.

### Step 19:

Server regenerates or adjusts the meal plan using the generative AI model.

Input: Secondary prompt sentence and existing meal plan context.

Output: An adjusted menu text that reflects nutrition and emotion constraints.

Server sends the secondary prompt to the generative AI model, receives the adjusted text, and replaces or merges specific days or dishes in the existing meal plan with the new recommendations, updating the meal plan repository accordingly.

### Step 20:

Server recomputes ingredients and nutrition after adjustment (if adjustment occurred).

Input: Adjusted structured meal plan object.

Output: Updated consolidated ingredient list and updated nutrition evaluation result.

Server re-executes the ingredient derivation, aggregation, and nutrition evaluation computations (as in Steps 14-16), compares new nutritional totals to thresholds, and verifies that constraints are now satisfied or within acceptable margins.

### Step 21:

Server maps consolidated ingredients to merchandise candidates.

Input: Final consolidated ingredient list and external merchandise information from an external merchandise information provision apparatus.

Output: A merchandise candidate list mapping each ingredient to one or more specific products with identifiers and package sizes.

Server sends ingredient names and required quantities to the external apparatus via an API, receives a catalog of matching items, filters by package size, unit price, and availability, scores each candidate based on a cost and waste minimization function, and selects the best product or set of products for each ingredient.

### Step 22:

Server constructs order information and delivery destination information.

Input: Merchandise candidate list, user profile including residence address, and possibly user delivery preferences.

Output: Structured order information containing product identifiers, quantities, price estimates, and structured delivery destination records.

Server compiles selected products into an order payload, attaches address information and preferred delivery windows, calculates estimated total cost and shipping fees, and formats the payload according to the protocol required by a target electronic commerce apparatus.

### Step 23:

Server submits the order to the electronic commerce apparatus.

Input: Order information and delivery destination information.

Output: An order confirmation response including an order identifier, acceptance status, and an initial delivery estimate.

Server sends the order payload over a secure API, handles authentication tokens, receives the response, verifies success or failure, logs the order identifier and status, and writes the data to the delivery status management store for tracking.

### Step 24:

Server initializes and updates delivery status tracking.

Input: Order confirmation details, including order identifier and initial delivery estimate, and subsequent status updates from the electronic commerce apparatus or logistics providers.

Output: A delivery status object that reflects current shipment state (e.g., “preparing,” “shipped,” “out for delivery,” “delivered”).

Server periodically polls external status endpoints or listens for webhook callbacks, parses status codes, timestamps, and tracking identifiers, updates the corresponding delivery record, and maintains a history of changes.

### Step 25:

Server sends final meal plan, ingredient list, nutrition information, and delivery status to the terminal.

Input: Final structured meal plan, consolidated ingredient list, nutrition evaluation summary, and current delivery status object.

Output: A unified response message transmitted to the terminal containing all relevant data for user presentation.

Server aggregates the data into a JSON response, associates descriptive labels for each field, and transmits the response to the terminal via a dedicated API endpoint.

### Step 26:

Terminal displays the results to the user.

Input: Unified response message containing meal plan, ingredient list, nutrition information, and delivery status.

Output: Graphical screens and components showing the weekly menu, shopping details, nutritional indicators, and order tracking information to the user.

Terminal renders days and dishes in a calendar or list format, presents ingredients with quantities and product names, displays nutrition indicators (such as icons or numeric values), and shows current delivery stage, expected arrival time, and tracking links.

### Step 27:

User reviews the plan and interacts with the displayed information.

Input: Visual representation of the meal plan, ingredient details, nutrition summary, and delivery status.

Output: User actions such as approval, modification requests, or feedback on specific dishes. User may accept the generated plan, request further changes to particular days or dishes, or provide satisfaction ratings; these actions can be sent back to the server for further processing or personalization in subsequent cycles.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

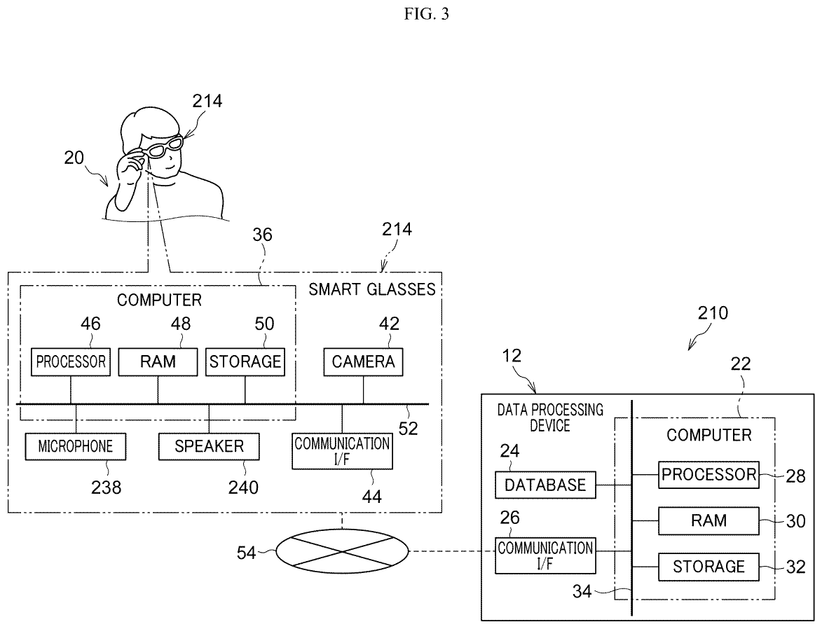

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

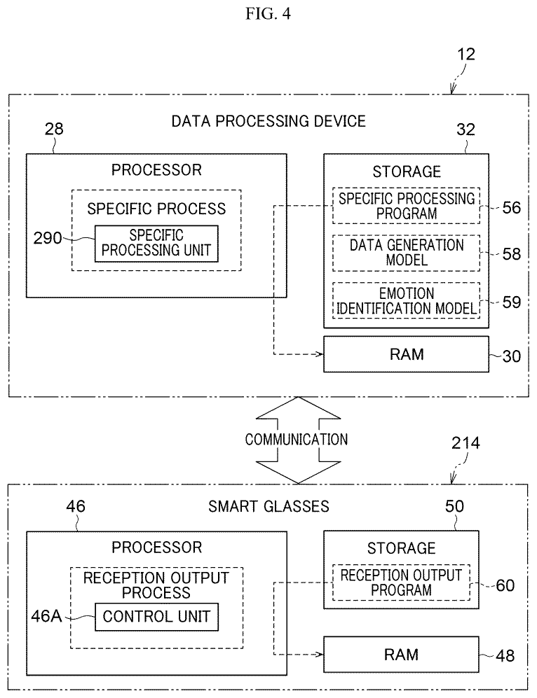

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

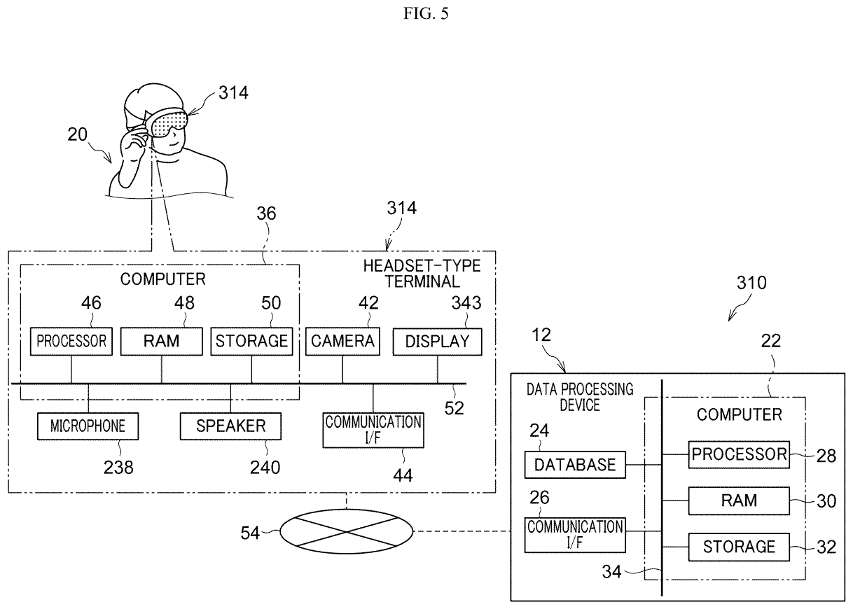

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

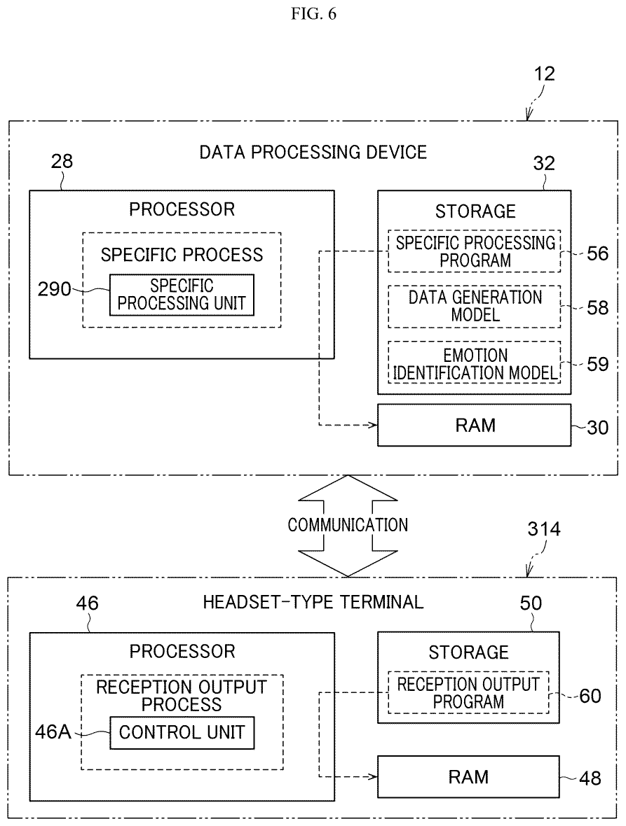

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

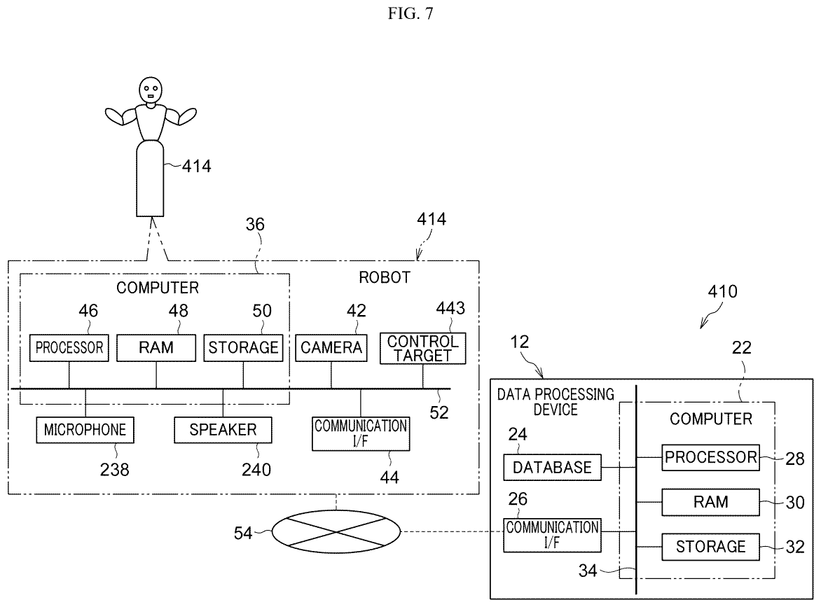

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

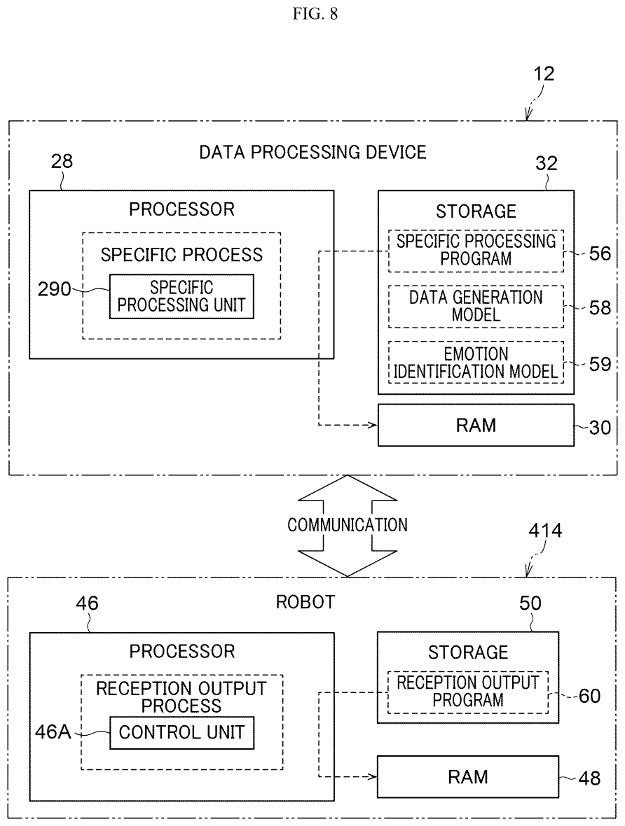

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

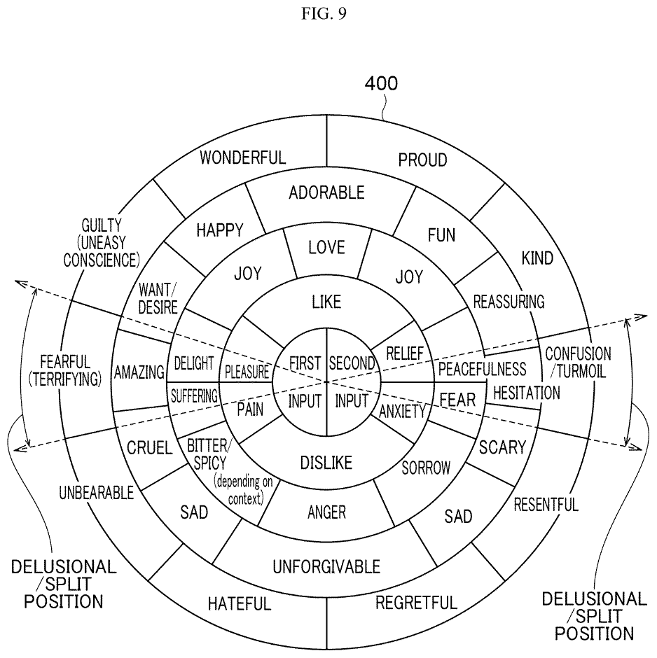

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

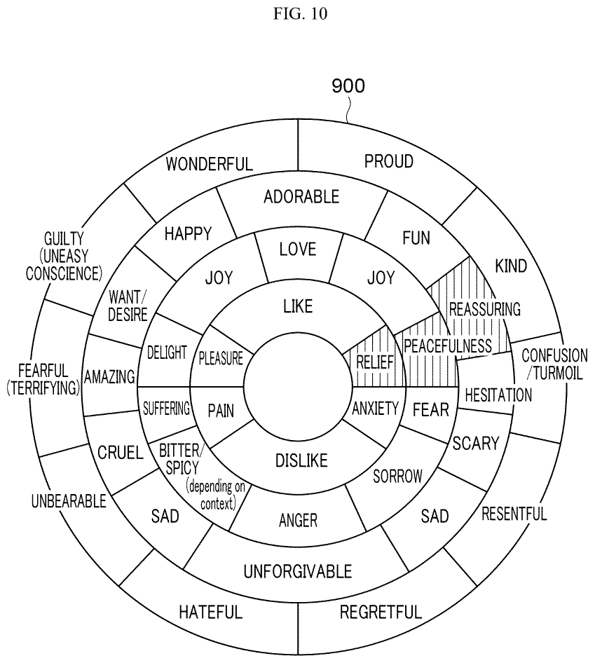

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor, a storage device, a communication interface, and a user interface device,
wherein the processor is configured to
acquire user condition information including budget information, number-of-persons information, and dietary-preference information through the user interface device as input and output means,
transmit the user condition information to an information processing apparatus through a communication path by using the communication interface, and cause meal-plan information transmitted from the information processing apparatus to be displayed on a display unit of the user interface device,
store, in the storage device, the received user condition information as structured information, and generate a natural-language prompt sentence based on the user condition information, the prompt sentence being intended for a generative AI model,
transmit, by using the communication interface, the prompt sentence to an external generative information processing model and acquire, from the generative information processing model, meal-plan information for a plurality of days generated based on the prompt sentence,
analyze the acquired meal-plan information to extract food-item information and quantity information, and aggregate the quantity information for each food item that appears across the plurality of meals to generate ingredient-list information,
generate, based on the ingredient-list information, order information including product-identification information and quantity information in a format required by an electronic commerce service information processing apparatus, and transmit the order information to the electronic commerce service information processing apparatus via a communication line by using the communication interface, and
store, in the storage device, order-acceptance information and delivery-schedule information received from the electronic commerce service information processing apparatus, and present the order-acceptance information and the delivery-schedule information to a user via the user interface device.

### (Supplementary 2)

The system according to supplementary 1,
wherein the processor is configured to
receive, via the user interface device, an approval input from the user with respect to the meal-plan information, and execute generation and transmission of the order information only when the approval input has been acquired, and inhibit generation and transmission of the order information when the approval input has not been acquired.

### (Supplementary 3)

The system according to supplementary 1,
wherein the processor is configured to
include, in the prompt sentence to be transmitted to the generative information processing model, condition information relating to an emotional state of the user, cause the generative information processing model to generate the meal-plan information adjusted in accordance with the emotional state of the user, and optimize proposal of the meal-plan information based on the emotional state.

### Application Example 1

### (Supplementary 1)

A system comprising a processor,
wherein the processor is configured to
provide, on a terminal, an input interface for receiving structured data including a user budget, a number of persons, dietary preferences, and nutritional constraints, and acquire the structured data from the terminal,
analyze the acquired structured data and generate a prompt sentence including conditions corresponding to the user budget, the number of persons, the dietary preferences, and the nutritional constraints, the prompt sentence being configured to instruct a generative AI model to generate a meal plan,
input the prompt sentence to the generative AI model and cause the generative AI model to perform numerical computation to generate a meal plan for a predetermined period based on the user budget, the number of persons, the dietary preferences, and the nutritional constraints,
extract, from the generated meal plan, information on a day basis and a meal basis, and calculate indices related to nutritional balance, energy intake, and ingredient composition, and evaluate whether the generated meal plan satisfies a nutritional balance condition, generate or update, for the generative AI model, an additional prompt sentence including information related to a user emotional state, and adjust contents, items, and quantities of the meal plan according to the user emotional state so as to optimize a proposal of the meal plan, configure an ingredient list corresponding to the generated meal plan, select, based on user location information and delivery conditions, a distribution base from among a plurality of sales entities, transmit order data of ingredients to an external information processing apparatus of a selected sales entity, and instruct delivery of the ingredients to a user residence location,
transmit the generated meal plan and the ingredient list to the terminal, and provide, on the terminal, a user interface for presenting the meal plan and receiving an approval input or a modification input from the user, and
execute the order data transmission and the delivery instruction of the ingredients only when an approval input of the meal plan is received from the terminal, and update order status and delivery status and notify the updated statuses to the terminal.

### (Supplementary 2)

The system according to supplementary 1,
wherein the processor is configured to perform a control in which the processor extracts, from an output meal plan generated by the generative AI model, a dish name, a meal category, an ingredient name, a quantity, and nutritional information by text analysis processing, stores extraction results as a data structure, calculates a cost of the ingredients based on the data structure, and regenerates the prompt sentence such that the calculated cost falls within a budget range of the user and re-inputs the regenerated prompt sentence to the generative AI model.

### (Supplementary 3)

The system according to supplementary 1,
wherein the processor is configured to learn a user preference pattern based on input history information, selection history information, and evaluation information acquired from the terminal, and reflect the learned preference pattern in the prompt sentence so as to adapt processing of meal plan generation by the generative AI model to each user.

### Example 2

### (Supplementary 1)

A system comprising a processor,
wherein the processor is configured to
receive budget information, number-of-persons information, and preference information from a user through an input interface, and accept the received budget information, number-of-persons information, and preference information as structured data, and
analyze the structured data, normalize the budget information, the number-of-persons information, and the preference information to convert them into an internal representation, and generate, based on the internal representation, a prompt sentence described in natural language that defines menu-generation conditions and an output format, and
transmit the prompt sentence as inquiry data to a generative AI model on an external or internal information processing platform, and receive text data output by the generative AI model in response to the prompt sentence, thereby obtaining menu information for a predetermined period, and
analyze the text data to extract, for each date within the predetermined period, dish information and ingredient information, and structure the extracted information as per-day menu data and ingredient-list data, and
normalize each ingredient name included in the ingredient-list data, integrate identical or similar ingredients, aggregate required quantities per ingredient, and generate a bulk-purchase ingredient list based on an aggregation result, and
refer, for each ingredient in the bulk-purchase ingredient list, to product information stored in product-information storage means to specify a sales product, and calculate a purchase quantity and an estimated total cost based on unit-price information and package-unit information of the sales product, and evaluate whether the estimated total cost conforms to the budget information, and
transmit the per-day menu data and the bulk-purchase ingredient list to a terminal device for display, accept an approval input or a modification-request input from the user, and, in a case where the approval input is received, transmit order data, based on the sales product and the purchase quantity, to a commercial-transaction system via a communication network and obtain delivery information, and
obtain a user emotional state by using an emotion acquisition interface or emotion analysis means, estimate the user emotional state, and, based on the emotional state and the per-day menu data, change content or configuration conditions of menus, and re-execute generation of the prompt sentence or re-execute an inquiry to the generative AI model for the changed menus so as to optimize menu proposals, and
refer, based on the menu information for the predetermined period, to nutrition-information storage means to estimate intake amounts for respective nutrients, evaluate whether a nutritional balance satisfies a predetermined condition, and, according to an evaluation result, adjust conditions of the prompt sentence and cause the generative AI model to regenerate the menus so as to obtain menu information that considers the nutritional balance.

### (Supplementary 2)

The system according to supplementary 1,
wherein the processor is configured to
execute transmission of the order data based on the per-day menu data and the bulk-purchase ingredient list, and acquisition of the delivery information, only when the approval input from the terminal device is received.

### (Supplementary 3)

The system according to supplementary 1,
wherein the processor is configured to
estimate the user emotional state by analyzing at least one of operation-history information on a user interface, input text information, and biometric information, and change preference conditions or constraint conditions included in the prompt sentence according to the estimated emotional state so as to dynamically optimize menu proposals by the generative AI model.

### Application Example 2

### (Supplementary 1)

A system comprising a processor,
wherein the processor is configured to
acquire, via an input interface, information including a user budget, a number of persons, a food preference, and information related to a user emotion,
normalize numerical information and character information included in the acquired information and convert the numerical information and the character information into a predetermined format,
specify an emotional state of the user based on the acquired character information and the information related to the user emotion,
automatically generate, from condition information including the user budget, the number of persons, the food preference, and the emotional state of the user, a prompt sentence for instructing a generative artificial intelligence model to generate a meal plan,
input the prompt sentence into the generative artificial intelligence model and cause the generative artificial intelligence model to perform character-string computation to generate meal plan information for a predetermined period,
analyze the generated meal plan information, extract ingredient information necessary for each meal over the predetermined period, and generate an ingredient list in which quantities of duplicate ingredients are aggregated and integrated,
refer to a nutrition information storage unit for each ingredient included in the ingredient list to acquire nutrition values, perform predetermined aggregation computation to evaluate a nutrition balance of the meal plan information as a whole, and regenerate or correct the meal plan information in accordance with an evaluation result,
reconstruct the prompt sentence based on the emotional state of the user and the evaluation result of the nutrition balance, reinput the reconstructed prompt sentence into the generative artificial intelligence model, and adjust the meal plan information so as to be adapted to the emotional state of the user and the nutrition balance,
finalize the ingredient list based on the adjusted meal plan information, control an information provision function of an external merchandise information provision apparatus to acquire merchandise information corresponding to the ingredient list, and select merchandise candidates from the acquired merchandise information,
transmit, in accordance with a predetermined communication procedure and based on the merchandise candidates and residence information of the user, order information for ingredients and delivery destination information to a nearest electronic commerce apparatus, and acquire information related to a delivery status, and
transmit the meal plan information, the ingredient list, information related to the nutrition balance, and the information related to the delivery status to a user terminal, and cause the user terminal to present the transmitted information to the user.

### (Supplementary 2)

The system according to supplementary 1,
wherein the processor is configured to acquire, from the user terminal, response information indicating an approval operation for the meal plan information by the user, and, when the approval operation is acquired, to transmit the order information for ingredients and the delivery destination information to the electronic commerce apparatus, and, when the approval operation is not acquired, to control a process such that the order is not executed.

### (Supplementary 3)

The system according to supplementary 1,
wherein the processor is configured to perform an emotion analysis computation using at least one of character information, voice information, and image information acquired from the user to classify the emotional state of the user, and to change, in accordance with the emotional state, a condition relating to a type of dish, a cooking load, a degree of luxury, or a health orientation in the prompt sentence, thereby causing the generative artificial intelligence model to generate meal plan information optimized for the emotional state of the user.

What is claimed is:

1. A system comprising:
circuitry configured to:
receive, via a communication interface coupled to a packet-switched network, input data from a terminal device, and apply an emotion identification model to the input data to estimate an emotion state of a user and generate emotion state data comprising an emotion category label and an emotion intensity score;
generate structured condition data by encoding user preference parameters and a quantity parameter derived from the input data together with the emotion category label and the emotion intensity score as constraint fields;
construct a prompt sentence by embedding the structured condition data into a natural language template, and execute inference processing using a generative neural network model with the prompt sentence as input to obtain multi-period plan data for a plurality of time periods;
apply a balance evaluation model to the multi-period plan data to compute a balance score across a plurality of evaluation dimensions, and regenerate the prompt sentence with adjusted constraint fields when the balance score falls below a balance threshold;
extract item identification data and quantity data from the multi-period plan data for each time period, and aggregate the quantity data by item identifier across all time periods to generate aggregated item data; and
store the structured condition data, the prompt sentence, the multi-period plan data, and the aggregated item data as record information in a storage device, and update generation conditions for subsequent prompt sentences based on the stored record information.

2. The system according to claim 1, wherein the circuitry is configured to apply the emotion identification model by extracting feature vectors from the input data using at least one of a text-based sentiment classification model and an acoustic feature extraction model, and computing the emotion category label and the emotion intensity score from the extracted feature vectors.

3. The system according to claim 2, wherein the circuitry is configured to select the natural language template from a plurality of templates stored in the storage device based on the emotion category label, and embed the emotion intensity score as a style weighting parameter in the prompt sentence to condition the generative neural network model to adjust plan content in accordance with the estimated emotion state.

4. The system according to claim 3, wherein the circuitry is configured to adjust the style weighting parameter toward a comfort emphasis value when the emotion category label indicates an elevated stress state, and toward a variety emphasis value when the emotion category label indicates a neutral or positive state.

5. The system according to claim 4, wherein the circuitry is configured to regenerate the prompt sentence by computing an adjusted constraint field specifying modified balance targets based on deficient evaluation dimensions identified by the balance evaluation model, and transmitting the regenerated prompt sentence to the generative neural network model to obtain updated multi-period plan data satisfying the balance threshold.

6. The system according to claim 1, wherein the circuitry is configured to extract the item identification data and the quantity data by applying a structured parsing algorithm to the multi-period plan data to identify item entries and associated quantity entries, and aggregate the quantity entries by item identifier to produce the aggregated item data.

7. The system according to claim 6, wherein the circuitry is configured to validate the aggregated item data against the quantity parameter to detect entries exceeding a quantity threshold, and regenerate the prompt sentence with an adjusted quantity constraint field when the quantity threshold is exceeded.

8. The system according to claim 7, wherein the circuitry is configured to generate request data comprising item identification data and quantity data derived from the aggregated item data in a format required by an external data service, receive an approval input from the terminal device, and transmit the request data to the external data service via the communication interface in response to the approval input.

9. The system according to claim 8, wherein the circuitry is configured to inhibit generation and transmission of the request data when the approval input is not received within a predetermined timeout period.

10. The system according to claim 8, wherein the circuitry is configured to store acceptance information and schedule information received from the external data service in the storage device, and transmit the acceptance information and the schedule information to the terminal device via the communication interface.

11. The system according to claim 10, wherein the circuitry is configured to receive status data from the external data service indicating availability of items in the aggregated item data, and regenerate the prompt sentence with substitution constraint fields for unavailable items.

12. The system according to claim 1, wherein the circuitry is configured to apply the balance evaluation model by computing a balance score for each of a plurality of evaluation dimensions derived from the multi-period plan data, and identifying evaluation dimensions having balance scores below respective dimension thresholds as deficient dimensions.

13. The system according to claim 12, wherein the circuitry is configured to include, in the regenerated prompt sentence, an adjusted constraint field for each identified deficient dimension specifying a modified target value, to cause the generative neural network model to generate updated multi-period plan data with improved balance scores in the deficient dimensions.

14. The system according to claim 1, wherein the circuitry is configured to update the generation conditions by computing updated preference parameter values from the stored record information including prior multi-period plan data and user modification inputs received from the terminal device, and incorporating the updated values into subsequent prompt sentences.

15. The system according to claim 14, wherein the circuitry is configured to compute a similarity score between a vector representation of a current prompt sentence and vector representations of stored record information entries, and retrieve entries having similarity scores exceeding a threshold value for incorporation as historical context data in a subsequent prompt sentence.

16. The system according to claim 1, wherein the circuitry is configured to transmit the multi-period plan data and the aggregated item data to the terminal device via the communication interface for display, and receive a modification input from the terminal device specifying adjustments to the multi-period plan data before any external transmission.

17. The system according to claim 16, wherein the circuitry is configured to update the aggregated item data based on the modification input, regenerate the prompt sentence incorporating the adjusted multi-period plan data, and store the modification input as record information in the storage device.

18. A system comprising:
circuitry configured to:
receive, via a communication interface coupled to a packet-switched network, input data from a terminal device, apply an emotion identification model to the input data to estimate an emotion state comprising an emotion category label and an emotion intensity score, and generate structured condition data encoding user preference parameters, a quantity parameter, the emotion category label, and the emotion intensity score as constraint fields;
select a natural language template from a plurality of templates stored in a storage device based on the emotion category label, construct a prompt sentence embedding the structured condition data and a style weighting parameter derived from the emotion intensity score, and execute inference processing using a generative neural network model to obtain multi-period plan data for a plurality of time periods;
apply a balance evaluation model to the multi-period plan data to compute a balance score across a plurality of evaluation dimensions, identify deficient dimensions having balance scores below respective thresholds, and regenerate the prompt sentence with adjusted constraint fields specifying modified balance targets for the deficient dimensions;
extract item identification data and quantity data from the multi-period plan data for each time period, aggregate the quantity data by item identifier across all time periods to produce aggregated item data, and validate the aggregated item data against the quantity parameter; and
transmit the multi-period plan data and the aggregated item data to the terminal device via the communication interface, and store the structured condition data, the prompt sentence, and the multi-period plan data as record information in the storage device for updating generation conditions of subsequent prompt sentences.

19. The system according to claim 18, wherein the circuitry is configured to generate request data comprising item identification data and quantity data derived from the aggregated item data in a format required by an external data service, receive an approval input from the terminal device, and transmit the request data to the external data service in response to the approval input, while inhibiting transmission when the approval input is not received within a predetermined timeout period.

20. A method comprising:
receiving, via a communication interface coupled to a packet-switched network, input data from a terminal device, and applying an emotion identification model to the input data to estimate an emotion state of a user and generate emotion state data comprising an emotion category label and an emotion intensity score;
generating structured condition data by encoding user preference parameters and a quantity parameter derived from the input data together with the emotion category label and the emotion intensity score as constraint fields;
constructing a prompt sentence by embedding the structured condition data into a natural language template, and executing inference processing using a generative neural network model with the prompt sentence as input to obtain multi-period plan data for a plurality of time periods;
applying a balance evaluation model to the multi-period plan data to compute a balance score across a plurality of evaluation dimensions, and regenerating the prompt sentence with adjusted constraint fields when the balance score falls below a balance threshold;
extracting item identification data and quantity data from the multi-period plan data for each time period, and aggregating the quantity data by item identifier across all time periods to generate aggregated item data; and
storing the structured condition data, the prompt sentence, the multi-period plan data, and the aggregated item data as record information in a storage device, and updating generation conditions for subsequent prompt sentences based on the stored record information.
